(19) United States
(12) Patent Application Publication
(10) Pub. No.: US 2024/0409644 A1
(43) Pub. Date: Dec. 12, 2024

(54) HUMANIZED ANTI-IL-1R3 ANTIBODIES

(71) Applicant: SANOFI BIOTECHNOLOGY, Gentilly (FR)

(72) Inventors: Stephan Fischer, Weilheim (DE); Michael Brandt, München (DE); Linda Veronique Kazandjian, Munich (DE)

(21) Appl. No.: 18/668,446

(22) Filed: May 20, 2024

Related U.S. Application Data
(63) Continuation of application No. 17/410,153, filed on Aug. 24, 2021, now Pat. No. 12,024,564.
Application No. 17/410,153 is a division of application No. 16/099,059, filed on Nov. 5, 2018, now Pat. No. 11,203,642.

(30) Foreign Application Priority Data
May 6, 2016 (EP) 16168617.5

(51) Int. Cl. C07K 16/28

(52) U.S. Cl. CPC C07K 16/2866; C07K 2317/24; C07K 2317/52; C07K 2317/76

(57) ABSTRACT

The present invention relates to humanized antibodies that specifically bind to IL-1R3 or a fragment or derivative thereof or a polypeptide that contains at least a portion of said antibody that is sufficient to confer IL-1R3 binding specificity. Said antibodies inhibit IL-1R3 induced NFkB activity. They also inhibit IL-1alpha, IL-1beta, IL-33, and/or IL-36 stimulated NFkB activity.

The invention further relates to use of said humanized antibody in the treatment of an IL-1R3 mediated disease such as cancer.

The present invention also encompasses a pharmaceutical composition comprising a pharmaceutically acceptable carrier and a therapeutically effective amount of the antibody according to the invention. The pharmaceutical composition can be used in treating a IL-1R3 mediated disease such as cancer.

### RELATED APPLICATIONS

This application is a continuation of U.S. patent application Ser. No. 17/410,153, filed Aug. 24, 2021, which is a divisional of U.S. patent application Ser. No. 16/099,059, filed Nov. 5, 2018, now U.S. Pat. No. 11,203,642, which is a 35 U.S.C. § 371 filing of International Patent Application No. PCT/EP2017/060925, filed May 8, 2017, which claims priority to European Patent Application No. 16168617.5, filed May 6, 2016, the entire disclosures of which are hereby incorporated herein by reference.

### SEQUENCE LISTING

The instant application contains a Sequence Listing which has been submitted electronically in XML format and is hereby incorporated by reference in its entirety. Said XML file, created on May 20, 2024, is named 745981_SA9-298USDIVCON_ST26.xml and is 226,053 bytes in size.

### FIELD OF INVENTION

The present invention relates to humanized anti-IL-1R3 antibodies and the use thereof.

### BACKGROUND

The interleukin 1 receptor accessory protein (IL1RAP), also called IL1R3, is a coreceptor of type 1 interleukin 1 receptor (IL1R1) and is indispensable for transmission of IL-1 signaling. Upon binding of IL-1, IL-1R1 associates with IL-1RAcP forming a functional signaling receptor complex, which stimulates NFkB activity.

IL-33, its receptor ST2, and IL-1RAcP form also a complex (IL-33/ST2/IL-1RAcP) with a similar activity in regard to NFkB activation as the IL-1β/IL-1R1/IL-1RAcP complex. IL-36 (IL-36α (IL-1F6), IL-36β (IL-1F8), and IL-36γ (IL-1F9)), their receptor IL-36R, and IL-1RAcP form also a complex (IL-36/II-36R/IL-1RAcP) with a similar activity in regard to NFkB activation as the IL-1β/IL-1R1/IL-1RAcP complex.

Human NF-kB is an important regulator of expression of several genes involved in inflammation, immune response and apoptosis. Therefore, the dysfunction of NFkB is involved in the in the pathology of various diseases, including autoimmune diseases, neurodegenerative diseases, inflammation, and cancers. For example, in the treatment of osteoarthritis (OA), the NF-kB pathway is an important target. Thus, agents that regulate the human NFkB pathway via inhibiting the signaling activity of the human IL-1R1/IL-1RAcP complex represent possible treatments for various human diseases. In particular, high affinity neutralizing antibodies would make excellent therapeutic agents.

Since more than 15 years, attempts have been made to generate functional monoclonal antibodies against human IL1RAcP. However, many attempts failed and existing antibodies still exhibit various drawbacks. WO199623067 relates to an IL-1RAcP antibody, which binds specifically to murine IL-1 receptor accessory protein. Examples 15 and 16 describe the attempt to generate anti-human IL-1RAcP antibodies, which neutralize IL-1 biological activity. However, no such antibody is provided by WO199623067 and example 16, describing an IL-1 induced IL-6 assay is only hypothetical. Do-Young Yoon D-Y and Charles A. Dinarello CA describe in J. Immunol. 1998; 160:3170-3179 polyclonal antibodies to domains II and III of the murine IL-1RAcP which inhibit IL-1beta activity but not binding. However, at higher concentrations of IL-1beta (1000 pg/ml), this polyclonal antiserum did not block the proliferation of D10S cells. (D10S is a subclone of the murine D10.G4.1 helper T-cell which proliferates to subfemtomolar (attomolar) concentrations of IL-1 beta or alpha in the absence of mitogens, cf. Orencole SF and Dinarello CA; Cytokine 1 (1989) 14-22). Jaras M. et al., PNAS 107 (2010) 16280-16285 describe the use of rabbit polyclonal anti-IL1RAP antibody KMT-1 for killing CML stems cell. This antibody induces ADCC in an IL1RAP-independent manner caused by its rabbit Fc part. Jaras et al. expect that “potential future therapeutic IL1RAP-targeting antibodies are expected to show low toxicity on normal hematopoietic cells”. Polyclonal rabbit antibodies against murine IL-1RAcP were also mentioned in Do-Young Yoon and Charles A. Dinarello, Journal of Biochemistry and Molecular Biology, Vol. 40, No. 4, July 2007, pp. 562-570.

WO2002064630 relates also to IL-1RAcP and its use, but no antibodies against IL-1RAcP are described. WO2004022718 and WO2009120903 mention theoretically that antibodies against CSF1R, IL13RA1, IL1RAP, IFNAR1, IL5R, INSR, IL1RL1, LTK, and TACSTD1 could be generated according to the state of the art. However, here also no antibody against IL-1RAcP is described. WO2011021014 and WO 2012098407 (U.S. Pat. No. 20,140,017167) relate to the polyclonal rabbit anti-human IL-1RAcP antiserum KMT-1 (see Jaras et al. 2010) and its use. WO2014100772 relates to an anti-IL-1RAcP antibody binding to IL-1RAcP. However, no activity in regard to inhibition of any functional signaling receptor complex (like IL-1β/IL-1R1/IL-1RAcP) which stimulates NFkB activity is described. U.S. Pat. No. 6,280,955 relates to IL-1RAcP and its use, but again no antibodies against IL-1RAcP are described. U.S. Pat. No. 7,390,880 mentions a N-terminal fragment of IL1RAP, but describe also no antibodies against IL-1RAcP.

WO2004100987 relates to the use of an interleukin-I (IL-1) antagonist in the preparation of a medicament for the treatment of neointimal hyperplasia and to the use of an IL-1 antagonist for the treatment of neointimal hyperplasia. As such an antagonist an anti-IL-1RAcP antibody is suggested but not further described. U.S. Pat. No. 20,030,26806 relates to antibodies binding to IL-1. WO2002064630 relates also to an IL-1 antagonist ant to IL-1RAcP protein. Though to the use of IL-1RAcP for screening for IL-1RAcP antagonists are mentioned, no such method or antagonist is disclosed.

This shows that it has been exceedingly difficult to identify monoclonal antibodies with high affinity, high specificity, and potent neutralizing activity against IL-1R3. The present invention encompasses humanized IL-1R3 antibody, with high affinity and specificity for IL-1R 3, with potent IL-1R3 neutralizing activity 3, and improved stability.

### SUMMARY OF INVENTION

The present invention relates to a humanized IgG1_{LALA} antibody that specifically binds to IL-1R3 or a fragment or derivative thereof or a polypeptide that contains at least a portion of said antibody that is sufficient to confer IL-1R3 binding specificity. Said antibody inhibits IL-1R3 induced NFkB activity. It also inhibits IL-1alpha, IL-1beta, IL-33, and/or IL-36 stimulated NFkB activity.

The invention further relates to an antibody according to the invention for the use in treating a IL-1R3 mediated disease.

The invention further encompasses a method of treating an IL-1R3 mediated disease in a patient, comprising administering to a patient a pharmaceutically effective amount of said antibody.

The present invention also comprises a pharmaceutical composition comprising a pharmaceutically acceptable carrier and a therapeutically effective amount of said antibody.

### DEFINITIONS

The term “rabbit” according to the invention means an animal of the members of the taxonomic order Lagomorpha, which includes the families (hares and rabbits) and Ochotonidae (pikas), preferably of genus Oryctolagus.

The term “antibody” encompasses the various forms of antibody structures including, but not being limited to, whole antibodies and antibody fragments as long as it shows the properties according to the invention.

The term “rabbit monoclonal antibody” according to the invention means a monoclonal antibody produced by immunizing a rabbit and isolated from a antigen producing cell of said rabbit as well as such an antibody which is further modified, preferably a humanized antibody, a chimeric antibody, a fragment thereof, or a further genetically engineered and recombinant produced antibody as long as the characteristic properties according to the invention are retained. Preferably the antibody is from a B cell or a rabbit hybridoma cell of said rabbit.

The term “antibody producing cell” according to the invention means a rabbit B cell which produce antibodies, preferably a B cell or rabbit hybridoma cell.

“Native antibodies” are usually heterotetrameric glycoproteins composed of two identical light (L) chains and two identical heavy (H) chains. Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (VH) followed by a number of constant domains. Each light chain has a variable domain at one end (VL) and a constant domain at its other end. The constant domain of the light chain is aligned with the first constant domain of the heavy chain, and the light-chain variable domain is aligned with the variable domain of the heavy chain. Particular amino acid residues are believed to form an interface between the light chain and heavy chain variable domains.

“Percent (%) amino acid sequence identity” with respect to a peptide or polypeptide sequence is defined as the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software.

The term “VL (or VH) region” has the same meaning as VL (or VH) domain.

The terms “Fc receptor” or “FcR” according to the invention refers to a human receptor that binds to the Fc region of an antibody. FcRs bind IgG antibodies and include receptors of the FcγRI, FcγRII, and FcγRIII subclasses, including allelic variants and alternatively spliced forms of these receptors. FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain (see review M. in Daeron, Annu. Rev. Immunol. 15:203-234 (1997)). FcRIIIA (CD16a) mediaties ADCC. FcRs are reviewed in Ravetch and Kinet, Annu. Rev. Immunol 9:457-92 (1991); Capel et al, Immunomethods 4:25-34 (1994); and de Haas et al, J. Lab. CHn. Med. 126:330-41 (1995). These and all other FcRs are encompassed by the term “FcR” herein. The term also includes the neonatal receptor, FcRn, which is responsible for the transfer of maternal IgGs to the fetus (Guyer et al, J. Immunol. 117:587 (1976) and Kim et al, J. Immunol. 24:249 (1994)) and mediates slower catabolismthus longer half-life.

The term “antibody effector function(s),” or “effector function” as used herein refers to a function contributed by an Fc effector domain(s) of an IgG (e.g., the Fc region of an immunoglobulin). Such function can be effected by, for example, binding of an Fc effector domain(s) to an Fc receptor on an immune cell with phagocytic or lytic activity or by binding of an Fc effector domain(s) to components of the complement system. Typical effector functions are ADCC, ADCP and CDC.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)2; diabodies; linear antibodies; single-chain antibody molecules (e.g. scFv); and multispecific antibodies formed from antibody fragments.

An “antibody that binds to the same epitope” as a reference antibody refers to an antibody that blocks binding of the reference antibody to its antigen in a competition assay by 50% or more, and conversely, the reference antibody blocks binding of the antibody to its antigen in a competition assay by 50% or more. An exemplary competition assay is provided herein.

“Antibody-dependent cell-mediated cytotoxicity” and “ADCC” refer to a cell-mediated reaction in which nonspecific cytotoxic cells that express FcRs (e.g. Natural Killer (NK) cells, neutrophils, and macrophages) recognize bound antibody on a target cell and subsequently cause lysis of the target cell. The primary cells for mediating ADCC, NK cells, express FcyRIII only, whereas monocytes express FcyRI, FcyRII and FCYRIII.

The term “Antibody-dependent cellular phagocytosis” and “ADCP” refer to a process by which antibody-coated cells are internalized, either in whole or in part, by phagocytic immune cells (e.g., macrophages, neutrophils and dendritic cells) that bind to an immunoglobulin Fc region.

C1q″ is a polypeptide that includes a binding site for the Fc region of an immunoglobulin. C1q together with two serine proteases, C1r and C1s, forms the complex C1, the first component of the complement dependent cytotoxicity (CDC) pathway. Human C1q can be purchased commercially from, e.g. Quidel, San Diego, Calif.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgGi, IgG₂, IgG₃, IgG₄, IgAi, and IgA₂. The heavy chain constant domains that correspond to the different classes of immunoglobulins are called α, δ, ε, γ, and μ, respectively.

An “effective amount” of an agent, e.g., a pharmaceutical formulation, refers to an amount effective, at dosages and for periods of time necessary, to achieve the desired therapeutic or prophylactic result.

The term “Fc region” herein is used to define a C-terminal region of an immunoglobulin heavy chain that contains at least a portion of the constant region. The term includes native sequence Fc regions and variant Fc regions. Unless otherwise specified herein, numbering of amino acid residues in the Fc region or constant region is according to the EU numbering system, also called the EU index, as described in Kabat, et al., Sequences of Proteins of Immunological Interest, 5th Ed. Public Health Service, National Institutes of Health, Bethesda, MD (1991).

A “variant Fc region” comprises an amino acid sequence which differs from that of a “native” or “wildtype” sequence Fc region by virtue of at least one “amino acid modification” as herein defined. The term “Fc-variant” as used herein refers to a polypeptide comprising a modification in an Fc domain. The Fc variants of the present invention are defined according to the amino acid modifications that compose them. Thus, for example, P329G is an Fc variant with the substitution of proline with glycine at position 329 relative to the parent Fc polypeptide, wherein the numbering is according to the EU index. The identity of the wildtype amino acid may be unspecified, in which case the aforementioned variant is referred to as P329G. For all positions discussed in the present invention, numbering is according to the EU index. The EU index or EU index as in Kabat or EU numbering scheme refers to the numbering of the EU antibody (Edelman, et al., Proc Natl Acad Sci USA 63 (1969) 78-85, hereby entirely incorporated by reference.) The modification can be an addition, deletion, or substitution. Substitutions can include naturally occurring amino acids and non-naturally occurring amino acids. Variants may comprise non-natural amino acids. Examples include U.S. Pat. No. 6,586,207; WO 98/48032; WO 03/073238; US 2004/0214988 Al; WO 05/35727 A2; WO 05/74524 A2; Chin, J.W., et al., Journal of the American Chemical Society 124 (2002) 9026-9027; Chin, J.W., and Schultz, P.G., ChemBioChem 11 (2002) 1135-1137; Chin, J.W., et al., PICAS United States of America 99 (2002) 11020-11024; and, Wang, L., and Schultz, P.G., Chem. (2002) 1-10, all entirely incorporated by reference.

The term “Fc region-containing polypeptide” refers to a polypeptide, such as an antibody or immunoadhesin (see definitions below), which comprises an Fc region.

The terms “Fc receptor” or “FcR” are used to describe a receptor that binds to the Fc region of an antibody. A FcR which binds an IgG antibody (a gamma receptor) includes receptors of the FcyRI, FcyRII, and FcyRIII subclasses, including allelic variants and alternatively spliced forms of these receptors. FcyRII receptors include FcyRIIA (an “activating receptor”) and FcyRIIB (an “inhibiting receptor”), which have similar amino acid sequences that differ primarily in the cytoplasmic domains thereof. Activating receptor FcyRIIA contains an immunoreceptor tyrosine-based activation motif (ITAM) in its cytoplasmic domain. Inhibiting receptor FcyRIIB contains an immunoreceptor tyrosine-based inhibition motif (ITIM) in its cytoplasmic domain, (see review in Daeron, M., Annu. Rev. Immunol. 15 (1997) 203-234). FcRs are reviewed in Ravetch, and Kinet, Annu. Rev. Immunol 9 (1991) 457-492; Capel, et al., Immunomethods 4 (1994) 25-34; and de Haas, et al., J. Lab. Clin. Med. 126 (1995) 330-41. Other FcRs, including those to be identified in the future, are encompassed by the term “FcR” herein. The term also includes the neonatal receptor, FcRn, which is responsible for the transfer of maternal IgGs to the fetus (Guyer, et al., J. Immunol. 117 (1976) 587 and Kim, et al., J. Immunol. 24 (1994) 249).

By “IgG Fc ligand” as used herein is meant a molecule, preferably a polypeptide, from any organism that binds to the Fc region of an IgG antibody to form an Fc/Fc ligand complex. Fc ligands include but are not limited to FcyRs, FcyRs, FcyRs, FcRn, Clq, C3, mannan binding lectin, mannose receptor, staphylococcal protein A, streptococcal protein G, and viral FcyR. Fc ligands also include Fc receptor homologs (FcRH), which are a family of Fc receptors that are homologous to the FcyRs (Davis, et al., Immunological Reviews 190 (2002) 123-136, entirely incorporated by reference). Fc ligands may include undiscovered molecules that bind Fc. Particular IgG Fc ligands are FcRn and Fc gamma receptors. By “Fc ligand” as used herein is meant a molecule, preferably a polypeptide, from any organism that binds to the Fc region of an antibody to form an Fc/Fc ligand complex.

By “Fc gamma receptor”, “FcyR” or “FcgammaR” as used herein is meant any member of the family of proteins that bind the IgG antibody Fc region and is encoded by an FcyR gene. In humans this family includes but is not limited to FcyRI (CD64), including isoforms FcyRIA, FcyRIB, and FcyRIC; FcyRII (CD32), including isoforms FcyRIIA (including allotypes H131 and R131), FcyRIIB (including FcyRIIB-I and FcyRIIB-2), and FcyRIIc; and FcyRIII (CD 16), including isoforms FcyRIIIA (including allotypes VI 58 and F158) and FcyRIIIb (including allotypes FcyRIIB-NAI and FcyRIIB-NA2) (Jefferis, et al., Immunol Lett 82(2002) 57-65, entirely incorporated by reference), as well as any undiscovered human FcyRs or FcyR isoforms or allotypes. An FcyR may be from any organism, including but not limited to humans, mice, rats, rabbits, and monkeys. Mouse FcyRs include but are not limited to FcyRI (CD64), FcyRII (CD32), FcyRIII (CD 16), and FCYRIII-2 (CD 16-2), as well as any undiscovered mouse FcyRs or FcyR isoforms or allotypes.

By “FcRn” or “neonatal Fc Receptor” as used herein is meant a protein that binds the IgG antibody Fc region and is encoded at least in part by an FcRn gene. The FcRn may be from any organism, including but not limited to humans, mice, rats, rabbits, and monkeys. As is known in the art, the functional FcRn protein comprises two polypeptides, often referred to as the heavy chain and light chain. The light chain is beta-2-microglobulin and the heavy chain is encoded by the FcRn gene. Unless otherwise noted herein, FcRn or an FcRn protein refers to the complex of FcRn heavy chain with beta-2-microglobulin.

An “immunoconjugate” means an antibody conjugated to one or more cytotoxic agents, such as a chemotherapeutic agent, a drug, a growth inhibitory agent, a toxin, another antibody or a radioactive isotope.

“Antibody fragments” comprise a portion of a full length antibody, preferably the variable regions thereof, or at least the antigen binding site thereof. Examples of antibody fragments include diabodies, Fab fragments, and single-chain antibody molecules. scFv antibodies are, e.g., described in Huston, J.S., Methods in Enzymol. 203 (1991) 46-88.

The terms “monoclonal antibody” or “monoclonal antibody composition” as used herein refer to a preparation of antibody molecules of a single amino acid composition.

The term “humanized antibody ” or “humanized version of an antibody” refers to antibodies in which a human variable region has been modified to comprise the CDRs of an antibody according to the invention. In a preferred embodiment, the CDRs of the VH and VL are grafted into the framework region of human antibody to prepare the “humanized antibody.” See e.g. Riechmann, L., et al, Nature 332 (1988) 323-327; and Neuberger, M.S., et al, Nature 314 (1985) 268-270. The heavy and light chain variable framework regions can be derived from the same or different human antibody sequences. The human antibody sequences can be the sequences of naturally occurring human antibodies. Human heavy and light chain variable framework regions are listed e.g. in Lefranc, M.-P., Current Protocols in Immunology (2000)—Appendix IP A.1P.1-A.1P.37 and are accessible via IMGT, the international ImMunoGeneTics information system® (http://imgt.cines.fr) or via http://vbase.mrc-cpe.cam.ac.uk.

The terms “specifically binding, against target, or anti-target antibody”, as used herein, refer to binding of the antibody to the respective antigen (target), measured by ELISA, wherein said ELISA preferably comprises coating the respective antigen to a solid support, adding said antibody under conditions to allow the formation of an immune complex with the respective antigen or protein, detecting said immune complex by measuring the Optical Density values (OD) using a secondary antibody binding to an antibody according to the invention and using a peroxidase-mediated color development.

The term “antigen” according to the invention refers to the antigen used for immunization or a protein comprising said antigen as part of its protein sequence. For example, for immunization a fragment of the extracellular domain of a protein (e.g. the first 20 amino acids) can be used and for detection/assay and the like the extracellular domain of the protein or the full length protein can be used.

The term “specifically binding” or “specifically recognized” herein means that an antibody exhibits appreciable affinity for an antigen and, preferably, does not exhibit significant cross-reactivity.

“Appreciable” binding affinity includes binding with an affinity of at least 10exp7M⁻¹, specifically at least 10exp8M⁻¹, more specifically at least 10exp9M⁻¹, or even more specifically at least 10exp10M⁻¹.

An antibody that “does not exhibit significant cross-reactivity” is one that will not appreciably bind to an undesirable other protein. An antibody specific for an epitope according to the invention will, for example, not significantly cross-react with other epitopes on IL-1R3. Specific binding can be determined according to any art-recognized means for determining such binding, e.g. by competitive binding assays (e.g. ELISA).

All protein terms as used herein refers to the human proteins. If a protein from another species is meant, this is explicitly mentioned.

The term “IL-1alpha” “, as used herein, refers to human IL-1 (UniProtKB P01583). The term “IL-1beta””, as used herein, refer to human IL-1beta (UniProtKB P01584). IL-1 stimulates thymocyte proliferation by inducing IL-2 release, B-cell maturation and proliferation, and fibroblast growth factor activity. IL-1 proteins are involved in the inflammatory response, being identified as endogenous pyrogens (UniProtKB).

The term “IL-33””, as used herein, refers to human IL-33 (UniProtKB 095760), a cytokine that binds to and signals through the IL1RL1/ST2 receptor which in turn activates NF-kappa-B and MAPK signaling pathways in target cells (UniProtKB).

The term “IL-36”, as used herein, refers to human IL-36alpha (UniProtKB Q9UHA7, IL-36beta (UniProtKB Q9NZH7) and or IL-36gamma (UniProtKB Q9NZH8). IL-36 are cytokines that bind to and signal through the IL1RL2/IL-36R receptor which in turn activates NF-kappa-B and MAPK signaling pathways in target cells linked to a pro-inflammatory response. IL-36 seems to be involved in skin inflammatory response by acting on keratinocytes, dendritic cells and indirectly on T cells to drive tissue infiltration, cell maturation and cell proliferation (UniProtKB).

The term “NFkB” as used herein, refer to human nuclear factor NF-kappa-B, which consists of p105 subunit (P19838) and p100 subunit (Q00653).

“Inhibition of NFkB” is measured according to the invention as inhibition of NFkB dependent luciferase gene expression in human cells. Such methods are e.g. described in Windheim M. et al., Mol. Cell. Biol. 28 (2008) 1783-1791; Huang J. et al. PNAS USA 94 (1997) 12829-12832; Xiaoxia L. et al., Mol. Cell, Biol. 19 (1999) 4643-4652. If murine NFkB is meant herein it is explicitly mentioned.

The “variable region (or domain) of an antibody according to the invention” (variable region of a light chain (VL), variable region of a heavy chain (VH)) as used herein denotes each of the pair of light and heavy chain regions which are involved directly in binding the antibody to the antigen. The variable light and heavy chain regions have the same general structure and each region comprises four framework (FR) regions whose sequences are widely conserved, connected by three complementary determining regions, CDRs. The antibody according to the invention comprises a VH region and a VL region or parts thereof, which are both together sufficient for the specific binding to the respective antigen.

The term “antigen-binding portion of an antibody” when used herein refer to the amino acid residues of an antibody which are responsible for antigen-binding. The antigen-binding portion of an antibody comprises preferably amino acid residues from the “complementary determining regions” or “CDRs”. The CDR sequences are defined according to Kabat et al, Sequences of Proteins of Immunological Interest, 5th Ed. Public Health Service, National Institutes of Health, Bethesda, Md. (1991). Using this numbering system, the actual linear amino acid sequence may contain fewer or additional amino acids corresponding to a shortening of, or insertion into, a FR or CDR of the variable region.

The phrases “parenteral administration” and “administered parenterally” as used herein means modes of administration other than enteral and topical administration, usually by injection, and includes, without limitation, intravenous, intramuscular, intra-arterial, intrathecal, intracapsular, intraorbital, intracardiac, intradermal, intraperitoneal, transtracheal, subcutaneous, subcuticular, intra-articular, subcapsular, subarachnoid, intraspinal, epidural and intrasternal injection and infusion.

The term “cancer” as used herein may be, for example, lung cancer, non-small cell lung (NSCL) cancer, bronchioloalviolar cell lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, gastric cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, cancer of the bladder, cancer of the kidney or ureter, renal cell carcinoma, carcinoma of the renal pelvis, mesothelioma, hepatocellular cancer, biliary cancer, neoplasms of the central nervous system (CNS), spinal axis tumors, brain stem glioma, glioblastoma multiforme, astrocytomas, schwanomas, ependymonas, medulloblastomas, meningiomas, squamous cell carcinomas, pituitary adenoma, lymphoma, lymphocytic leukemia, including refractory versions of any of the above cancers, or a combination of one or more of the above cancers.

### DETAILED DESCRIPTION OF THE INVENTION

The humanization of antibodies that originate from immunized mammals is required as quality feature in development, when said antibodies are meant for use as human therapeutics. Goal of the humanization is to keep the original characteristics (binding specificity, activity) of the antibody as far as possible, while reducing the immunological side effects that may occur when introducing non-humanized antibodies derived from other organisms, into humans. The present invention is based on the known humanization strategy of CDR grafting. Here, a large amount of active humanized antibodies was produced in parallel and top candidates were chosen for further assessment.

As outlined in the introduction of this application, it is exceedingly difficult to identify monoclonal antibodies with high affinity, high specificity, and potent neutralizing activity against IL-1R3. The present invention encompasses humanized IL-1R3 antibody, with high affinity and specificity for IL-1R 3, with potent IL-1R3 neutralizing activity 3, and improved stability.

In particular, the present invention relates to humanized antibody that specifically binds to IL-1R3 or a fragment or derivative thereof that contains at least a portion of said antibody that is sufficient to confer IL-1R3 binding specificity, comprising:
a) a heavy chain variable region (VH) comprising the complementary determining regions comprising CDR-H1, CDR-H2, and CDR-H3
   wherein the CDR-H1 region comprises an amino acid sequence selected from the group of SEQ ID NO: 69-85,
   wherein the CDR-H2 region comprises an amino acid sequence selected from the group of SEQ ID NO: 86-102,
   and wherein the CDR-H3 region comprises an amino acid sequence selected from the group of SEQ ID NO: 103-119; and
b) a light chain variable region (VL) comprising the complementary determining regions comprising CDR-L1, CDR-L2, and CDR-L3
   wherein the CDR-L1 region comprises an amino acid sequence selected from the group of SEQ ID NO: 120-136,
   wherein the CDR-L2 region comprises an amino acid sequence selected from the group of SEQ ID NO: 137-153, and
   wherein the CDR-L3 region comprises an amino acid sequence selected from the group of SEQ ID NO: 154-170 and 175.

In one embodiment, the antibody of the invention comprises a substitution at position 2 of CDR-L3. Said substitution may be a cysteine to serine substitution.

Moreover, the present invention encompasses a humanized antibody that specifically binds to IL-1R3 or a fragment or derivative thereof that contains at least a portion of said antibody that is sufficient to confer IL-1R3 binding specificity, wherein the antibody comprises a heavy chain variable (VH) region that is least 60% identical, preferably at least 70% identical, more preferably at least 80% identical, more preferably at least 90% identical to a VH region selected from the group consisting of VH regions of SEQ ID NO: 1 to 34 and SEQ ID NO:173.

In one embodiment, the humanized antibody that specifically binds to IL-1R3 or a fragment or derivative thereof that contains at least a portion of said antibody that is sufficient to confer IL-1R3 binding specificity, comprises a heavy chain variable region (VH) sequence having at least 90%, 91%, 92%, 93%, 94%, 95%, 96%, 97%, 98%, 99%, or 100% sequence identity to an amino acid sequence selected from the group of VH sequences according to the invention.

In certain embodiments, a VH sequence having at least 90%, 91%, 92%, 93%, 94%, 95%, 96%, 97%, 98%, or 99% identity contains substitutions (e.g., conservative substitutions), insertions, or deletions relative to the reference sequence, whereby the antibody retains the ability to bind specifically according to the invention to the respective antigen.

The present invention also relates to a humanized antibody that specifically binds to IL-1R3 or a fragment or derivative thereof that contains at least a portion of said antibody that is sufficient to confer IL-1R3 binding specificity, wherein the antibody comprises a light chain variable (VL) region that is least 60% identical, preferably at least 70% identical, more preferably at least 80% identical, more preferably at least 90% identical to a VL region selected from the group consisting of VL regions of SEQ ID NO: 35 to 68 and 174.

In another embodiment the humanized antibody that specifically binds to IL-1R3 or a fragment or derivative thereof that contains at least a portion of said antibody that is sufficient to confer IL-1R3 binding specificity, comprises a light chain variable region (VL) having at least 90%, 91%, 92%, 93%, 94%, 95%, 96%, 97%, 98%, 99%, or 100% sequence identity to the amino acid sequence of the VL sequences according to the invention.

In certain embodiments, a VL sequence having at least 90%, 91%, 92%, 93%, 94%, 95%, 96%, 97%, 98%, or 99% identity contains substitutions (e.g., conservative substitutions), insertions, or deletions relative to the reference sequence, whereby the antibody retains the ability to bind specifically to the respective antigen.

In certain embodiments, a total of 1 to 10 amino acids have been substituted, inserted and/or deleted in said VL sequences. In certain embodiments, the substitutions, insertions, or deletions occur in regions outside the CDRs (i.e., in the FRs). The invention also comprises affinity matured antibodies which can be produced according to methods known in the art. Marks et al. Bio/Technology 10:779-783 (1992) describes affinity maturation by VH and VL domain shuffling. Random mutagenesis of CDR and/or framework residues is described by: Barbas et al., Proc Nat. Acad. Sci, USA 91: 3809-3813 (1994); Schier et al., Gene 169: 147-155 (1995); Yelton et al., J. Immunol. 1 55: 1994-2004 (1995); Jackson et al., J. Immunol. 1 54(7):3310-9 (1995); and Hawkins et al., J. Mol. Biol. 226:889-896 (1992) and WO2010108127.

In certain embodiments, a total of 1 to 10 amino acids have been substituted, inserted and/or deleted in each of said VH or VL sequences. In one embodiment, the antibody of the invention comprises a substitution at position 90 of the VH or VL sequence. It is preferred that the amino acid at position 90 is substituted by a serine. This substitution is preferably at position 90 of the light chain variable region (VL). In a preferred embodiment, the cysteine at position 90 of SEQ ID. NO: 62 is substituted by a serine. However, the antibodies of this invention are not limited to an amino acid substitution at position 90 but may comprise any substitution, deletion or insertion that leads to a functional antibody possessing the properties of the antibodies of this invention.

Therefore, the VL and VH sequences of the antibodies of this invention may also comprise further mutations at different positions.

In certain embodiments, substitutions, insertions, or deletions occur in regions outside the CDRs (i.e., in the FRs).

In other embodiments, the substitutions, insertions, or deletions occur in regions inside the CDRs. In one preferred embodiment, the antibody of the invention comprises a substitution at position 2 of CDR-L3. It is preferred that this substitution is cysteine to serine. In one embodiment, said substitution is in SEQ ID NO: 164.

The present invention also encompasses a humanized antibody that specifically binds to IL-1R3 or a fragment or derivative thereof that contains at least a portion of said antibody that is sufficient to confer IL-1R3 binding specificity, wherein the antibody comprises a heavy chain variable region (VH) comprising an amino acid sequence selected from the group of SEQ ID NO: 1 to 34 and 173.

Preferably, the heavy chain variable region (VH) sequence is SEQ ID NO:1, alternatively SEQ ID NO:2, or SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, or SEQ ID NO:29, SEQ ID NO:30, SEQ ID NO:31, SEQ ID NO:32, SEQ ID NO:33, or alternatively SEQ ID NO:34 or 173.

Furthermore, the invention relates to a humanized antibody that specifically binds to IL-1R3 or a fragment or derivative thereof that contains at least a portion of said antibody that is sufficient to confer IL-1R3 binding specificity, wherein the antibody comprises a light chain variable region (VL) comprising an amino acid sequence selected from the group of SEQ ID NO: 35 to 68 and 174.

Even more preferred, the light chain variable region (VL) sequence is SEQ ID NO:35, or SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, or alternatively SEQ ID NO:68 or 174.

The humanized antibody according to the invention that specifically binds to IL-1R3 or a fragment or derivative thereof that contains at least a portion of said antibody that is sufficient to confer IL-1R3 binding specificity, also comprises a VH region and a VL region comprising the respective CDR1, CDR2 and CDR3 regions of an antibody selected from the group consisting of MAB-15-0139, MAB-15-0106MAB-15-0108, MAB-15-0110, MAB-15-0117, MAB-15-0121, MAB-15-0140, MAB-15-0115, MAB-15-0125, MAB-15-0119, MAB-15-0109, MAB-15-0097, MAB-15-0135, MAB-15-0133, MAB-15-0107, MAB-15-0128, MAB-15-0116, MAB-16-0004, MAB-16-0009, MAB-16-0028, MAB-16-0031, MAB-16-0043, MAB-16-0049, MAB-16-0045, MAB-16-0040, MAB-16-0036, MAB-16-0046, MAB-16-0030, MAB-16-0021, MAB-16-0019, MAB-16-0015, MAB-16-0027, MAB-16-0048, MAB-16-0041, MAB-16-0149, MAB-16-0150.

In one embodiment, the humanized antibody that specifically binds to IL-1R3 or a fragment or derivative thereof that contains at least a portion of said antibody that is sufficient to confer IL-1R3 binding specificity, comprises SEQ ID NO.: 1 and 35, or SEQ ID NO.: 2 and 36. An antibody according to the invention may also comprise SEQ ID NO.: 3 and 37, or SEQ ID NO.: 4 and 38, or SEQ ID NO.: 5 and 39., or SEQ ID NO.: 6 and 40., or SEQ ID NO.: 7 and 41., or SEQ ID NO.: 8 and 42, or SEQ ID NO.: 9 and 43, or SEQ ID NO.: 10 and 44, or SEQ ID NO.: 11 and 45, or SEQ ID NO.: 12 and 46. Alternatively, an antibody according to the invention comprises SEQ ID NO.: 13 and 47, or SEQ ID NO.: 14 and 48, or SEQ ID NO.: 15 and 49, or SEQ ID NO.: 16 and 50, or SEQ ID NO.: 17 and 51, or SEQ ID NO.: 18 and 52, or SEQ ID NO.: 19 and 53, or SEQ ID NO.: 20 and 54, or SEQ ID NO.: 21 and 55, or SEQ ID NO.: 22 and 56, or SEQ ID NO.: 23 and 57, or SEQ ID NO.: 24 and 58, or SEQ ID NO.: 25 and 59, or SEQ ID NO.: 26 and 60, or SEQ ID NO.: 27 and 61.

Alternatively, an antibody according to the invention comprises SEQ ID NO.: 28 and 62, or SEQ ID NO.: 29 and 63, or SEQ ID NO.: 30 and 64, or SEQ ID NO.: 31 and 65, or SEQ ID NO.: 32 and 66, or SEQ ID NO.: 33 and 67, or SEQ ID NO.: 34 and 68.

Most preferably, the humanized antibody that specifically binds to IL-1R3 or a fragment or derivative thereof that contains at least a portion of said antibody that is sufficient to confer IL-1R3 binding specificity comprises the constant region sequences CR-H (SEQ ID NO. 172) and CR-L (SEQ ID NO. 171) and a VH region selected from the group of SEQ ID NO: 1 to 34 and 173 and a VL region selected from the group of SEQ ID NO: 35 to 68 and 174.

The humanized antibody that specifically binds to IL-1R3 or a fragment or derivative thereof that contains at least a portion of said antibody that is sufficient to confer IL-1R3 binding specificity, also comprises the constant region sequences CR-H (SEQ ID NO. 172) and CR-L (SEQ ID NO. 171) and a VH region and a VL region comprising the respective CDR1, CDR2 and CDR3 regions of an antibody selected from the group consisting of MAB-15-0139, MAB-15-0106, MAB-15-0108, MAB-15-0110, MAB-15-0117, MAB-15-0121, MAB-15-0140, MAB-15-0115, MAB-15-0125, MAB-15-0119, MAB-15-0109, MAB-15-0097, MAB-15-0135, MAB-15-0133, MAB-15-0107, MAB-15-0128, MAB-15-0116, MAB-16-0004, MAB-16-0009, MAB-16-0028, MAB-16-0031, MAB-16-0043, MAB-16-0049, MAB-16-0045, MAB-16-0040, MAB-16-0036, MAB-16-0046, MAB-16-0030, MAB-16-0021, MAB-16-0019, MAB-16-0015, MAB-16-0027, MAB-16-0048, MAB-16-0041, MAB-16-0149 and MAB-16-150.

According to the preferred therapeutic application of the antibodies according to the invention, the effector functions (such as ADCC) of the antibodies of the invention are reduced or lacking. In contrast to other antibodies of prior art, such as CAN04 (e.g. WO 2015/132602 A1), the antibodies of the invention do not exhibit increased effector functions and do not include ADCC.

Preferably, the humanized antibodies according to the invention show reduced or no Fcγ-receptor signaling.

Therefore, the invention also relates to a humanized antibody, wherein said antibody comprises at least amino acid substitutions at L234A and L235A of the human IgG1 Fc region or S228P and L235E of the human IgG4 Fc region.

In one embodiment according to the invention, the antibody is a humanized IgG1_{LALA} antibody.

In one embodiment according to the invention, a humanized humanized antibody that specifically binds to IL-1R3 or a fragment or derivative thereof that contains at least a portion of said antibody that is sufficient to confer IL-1R3 binding specificity, inhibits IL-1R3 induced NFkB activity.

In another embodiment, the humanized antibody that specifically binds to IL-1R3 or a fragment or derivative thereof that contains at least a portion of said antibody that is sufficient to confer IL-1R3 binding specificity, binds to the same epitope as an antibody selected from the group of antibodies MAB-15-0139, MAB-15-0106, MAB-15-0108, MAB-15-0110, MAB-15-0117, MAB-15-0121, MAB-15-0140, MAB-15-0115, MAB-15-0125, MAB-15-0119, MAB-15-0109, MAB-15-0097, MAB-15-0135, MAB-15-0133, MAB-15-0107, MAB-15-0128, MAB-15-0116, MAB-16-0004, MAB-16-0009, MAB-16-0028, MAB-16-0031, MAB-16-0043, MAB-16-0049, MAB-16-0045, MAB-16-0040, MAB-16-0036, MAB-16-0046, MAB-16-0030, MAB-16-0021, MAB-16-0019, MAB-16-0015, MAB-16-0027, MAB-16-0048, MAB-16-0041, MAB-16-0149 and MAB-16-150.

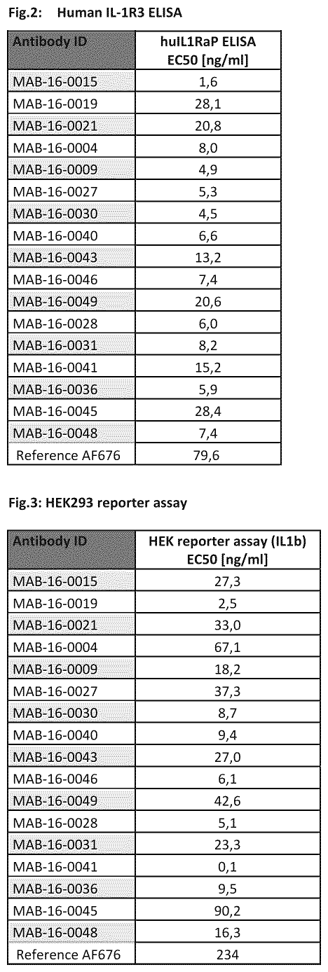

The antibodies according to the invention have the advantage to be very potent when it comes to binding to their target. They exhibit a strong binding capacity to their antigen, IL1R3, but not to other receptors. The binding properties of the antibodies were studied in biochemical enzyme-linked immunosorbent assays (ELISA) and cell binding analysis (flow cytometry) and are exemplified in FIGS. 2, 6 and 7.

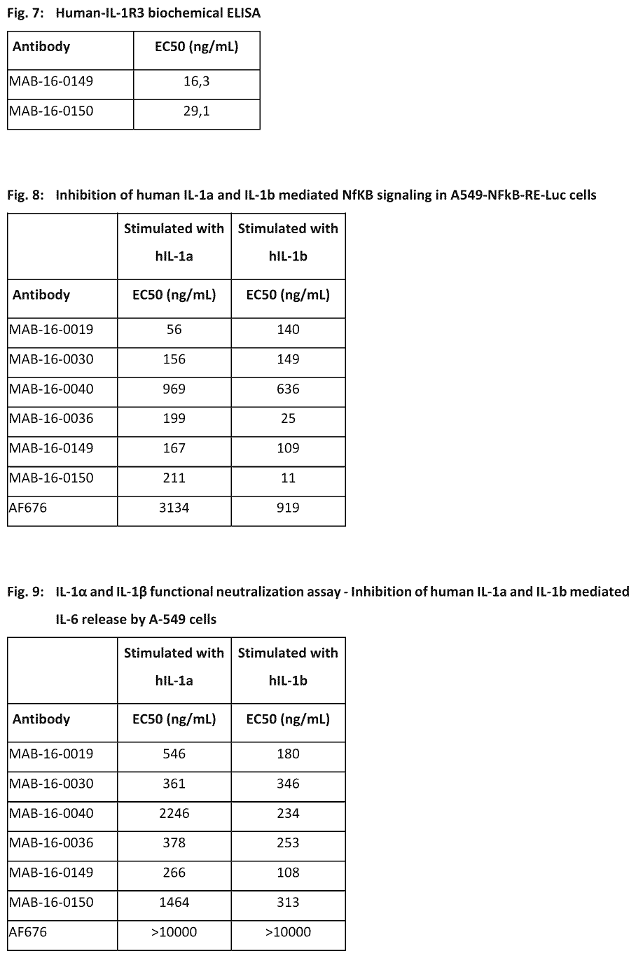

Preferred antibodies according to the invention, show a half maximal effective concentration (EC50) of less than 30 ng/ml, preferably of less than 20 ng/ml. In other embodiments, they show an EC50 of less than 15 ng/ml, 10 ng/ml or of less than 5 ng/ml. A preferred antibody according to the invention shows an EC50 of 16.3 ng/ml in a biochemical ELISA experiment (cf. FIG. 7).

The antibodies according to the invention also show a very strong binding to their antigen in experiments in which human IL1R3 is expressed in different cell lines while the antibodies do not bind cell lines not expressing human IL1R3 (e.g. NIH-3T3, cf. FIG. 5).

In the IL1R3 high-expressing cell line SK-MEL-30 (cf. FIG. 6, Example 8) the antibodies exhibit an EC50 of preferably less than 400 ng/ml, more preferably less than 350 ng/ml, or less than 310 ng/ml.

In one preferred embodiment encompassed by the invention, the antibody according to the invention inhibits IL-1alpha and/or IL-1beta stimulated NFkB activity. FIGS. 3, 4 and 8 exemplify the strong inhibitory activity of the antibodies according to the invention.

In one embodiment, the humanized antibody that specifically binds to IL-1R3 or a fragment or derivative thereof that contains at least a portion of said antibody that is sufficient to confer IL-1R3 binding specificity, inhibits IL-1alpha stimulated NFkB activity.

In another embodiment, the humanized antibody that specifically binds to IL-1R3 or a fragment or derivative thereof that contains at least a portion of said antibody that is sufficient to confer IL-1R3 binding specificity inhibits IL-1beta stimulated NFkB activity.

It is preferred that an antibody according to the invention inhibits IL-1beta stimulated NFkB activity in HEK293T/17-FR cells with an EC50 of less than 100 ng/ml, preferably of less than 95 ng/ml, 85 ng/ml, 75 ng/ml, 65 ng/ml, 55 ng/ml, 45 ng/ml, 35 ng/ml, 25 ng/ml, 20 ng/ml and most preferred of less than 15 ng/ml (e.g. cf . FIG. 3).

It is further preferred that an antibody according to the invention inhibits IL-1alpha stimulated NFkB activity with an EC50 of less than 1000 ng/ml, preferably of less than 500 ng/ml, 300 ng/ml, 200 ng/ml, and most preferred of less than 100 ng/ml (e.g. cf. FIG. 8) in A549-NFkB-RE-Luc cells.

It is further preferred that an antibody according to the invention inhibits IL-1beta stimulated NFkB activity with an EC50 of less than 700 ng/ml, preferably of less than 600 ng/ml, 300 ng/ml, 200 ng/ml, 100 ng/ml and most preferred of less than 50 ng/ml in A549-NFkB-RE-Luc cells (e.g. cf. FIG. 8).

The invention also encompasses a humanized antibody, wherein said antibody inhibits NFkB activity stimulated by a complex selected from the group consisting of IL-1B/IL-1R1/IL-1RACP, IL-1a/IL-1R1/IL-1RAcP IL-33/ST2/IL-1RAcP, and/or IL-36/II-36R/IL-1RAcP.

Moreover, a humanized antibody according to invention inhibits in a concentration of 10 μg/ml (rabbit IgG isotype has a molecular weight of 150 KD) NFkB expression in A549-NFkB-RE-Luc cell lysates (Steady-Glo™ Luciferase Assay System; Promega; Cat. No. E2510) stimulated with 0.1 ng/ml human IL-1alpha, human IL-1beta, IL-33 and/or IL-36 (molecular weight see UniProtKB/Swiss-Prot), for 50% or more, preferably for 70% or more, preferably for 80% or more preferably for 90% and more, and more preferably for 95% or more, related to the same assay without said antibody according to the invention.

In one embodiment, the humanized antibody according to the invention inhibits IL-1alpha, IL-1beta, IL-33,and/or IL-36, respectively, stimulated luciferase activity in HEK 293T/17 cells (HEK 293T/17-FR cells transfected with luciferase under control of NF-kB reporter gene), HEK-Blue-IL33™ cells (Invivogen) or HEK-293/17-IF cells.

Preferably, said IL-1alpha, stimulated luciferase activity is inhibited by 50% or more, preferably by 70% or more, preferably by 80% or more, preferably by 90% and more, and more preferably by 95% or more. Preferably, said IL-1alpha, stimulated luciferase activity is inhibited by 95%.

Preferably, said IL-1beta, stimulated luciferase activity is inhibited by 50% or more, preferably by 70% or more, preferably by 80% or more, preferably by 90% and more, and more preferably by 95% or more. Preferably, said IL-1beta, stimulated luciferase activity is inhibited by 95%.

Preferably, said IL-33, stimulated luciferase activity is inhibited by 50% or more, preferably by 70% or more, preferably by 80% or more, preferably by 90% and more, and more preferably by 95% or more. Preferably, said IL-33, stimulated luciferase activity is inhibited by 95%.

Preferably, said IL-36, stimulated luciferase activity is inhibited by 50% or more, preferably by 70% or more, preferably by 80% or more, preferably by 90% and more, and more preferably by 95% or more. Preferably, said IL-36, stimulated luciferase activity is inhibited by 95%.

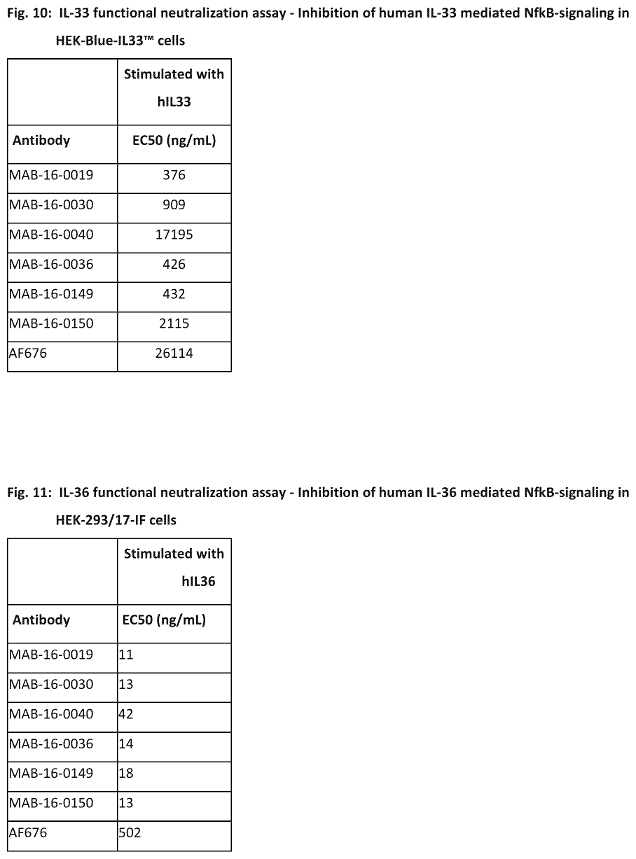

Furthermore, the antibodies according to the invention inhibit human IL-1a and IL-1b mediated IL-6 release and are superior to polyclonal antibodies. This potent inhibitory activity is shown and exemplified in FIG. 9. In these experiments, the EC50 values demonstrate that humanized anti-IL-1R3 IgG1-LALA antibodies are superior to that of goat-anti-human-IL1-R3 polyclonal antibody AF676 (R&D Systems). In preferred embodiments of the invention, the antibodies inhibit human IL-a mediated IL-6 release with an EC50 of less than 2500 ng/ml, preferably of less than 1500 ng/ml, less than 1000 ng/ml, less than 600 ng/ml, less than 400 ng/ml, or less than 300 ng/ml. It is also preferred that the antibodies of the invention inhibit human IL-1b mediated IL-6 release with an EC50 of less than 500 ng/ml, preferably of less than 400 ng/ml, less than 300 ng/ml, less than 200 ng/ml, or less than 150 ng/ml.

In another embodiment according to the invention, the antibodies inhibit human IL-33 mediated NfkB-signaling. FIG. 10 exemplifies the inhibitory activity of selected antibodies according to the invention in HEK-Blue-IL33™ cells and demonstrates the superiority over polyclonal antibodies. In preferred embodiments of the invention, the antibodies inhibit human IL-33 mediated NfkB-signaling with an EC50 of less than 20000 ng/ml, preferably of less than 18000 ng/ml, less than 3000 ng/ml, less than 1000 ng/ml, less than 500 ng/ml, or less than 400 ng/ml.

The antibodies of the invention may also inhibit human IL-36 mediated NfkB-signaling (FIG. 11). Preferably, they inhibit human IL-36 mediated NfkB-signaling at an EC50 of less than 100 ng/ml, preferably at less than 50 ng/ml, less than 40 ng/ml, less than 30 ng/ml, less than 20 ng/ml, or less than 15 ng/ml.

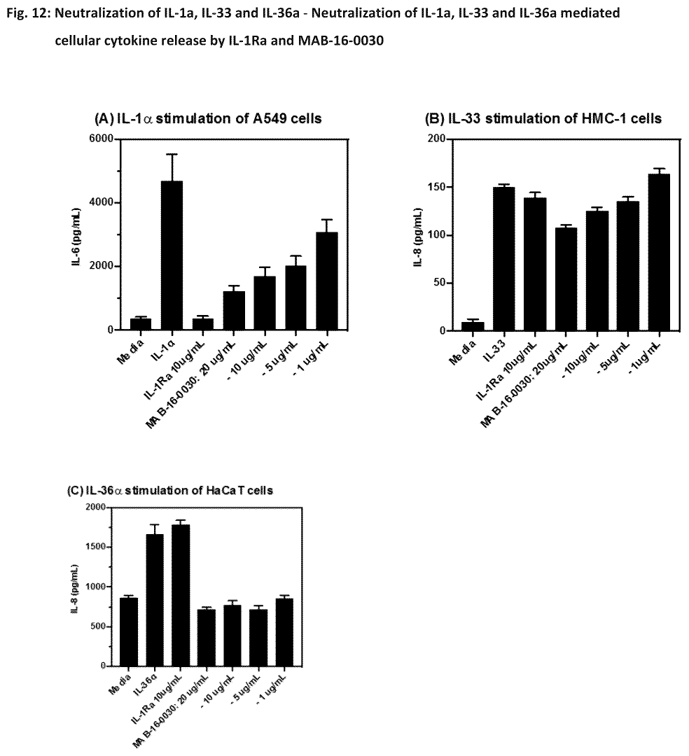

Strikingly, the inventors found that the antibodies according to the invention inhibit cytokine release mediated by various different stimuli. For example, the antibodies inhibit cytokine release mediated by IL-1a, IL-33 and IL-36a. Results of a selected antibody are shown in FIG. 12. For example, the antibody MAB-16-0030 inhibits cytokine release mediated by all three stimuli, while IL-1Ra affects only IL-1a mediated cytokine release.

Diseases associated with acute or chronic inflammation are maintained or establish by the action of multiple cytokines either at the same time or consecutively. Early “alarmins” such as IL-1a and IL-33 may trigger other cytokines including IL-1b and IL-36 to establish a strong inflammatory environment. Therefore, the concomitant inhibition of signaling mediated by multiple cytokines exerts efficacious control of inflammatory processes. It is a key aspect of the antibodies of the invention that they inhibit multi-cytokine signaling via the blockage of the IL1R3 receptor.

Binding of antibodies to immune cells may result in cell depleting and deleterious effects, e.g. by direct induction of apoptotic signaling pathways, stimulation of excessive cytokine release or antibody effector function mediated antibody dependent cellular cytotoxicity antibody (ADCC).

Accordingly, it is one further aspect of the antibodies of the invention that they do not induce immune cell depletion, mediated by direct induction of apoptotic signaling pathways, stimulation of excessive cytokine release or antibody effector function mediated antibody dependent cellular cytotoxicity.

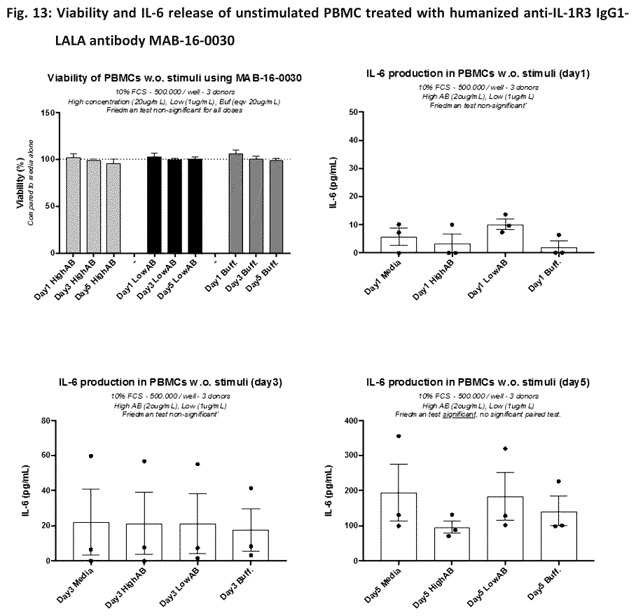

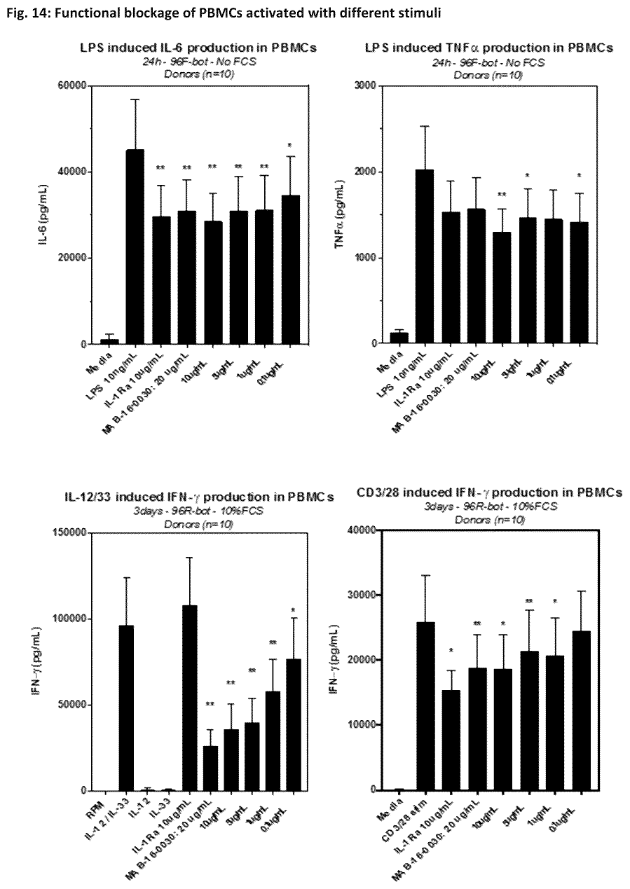

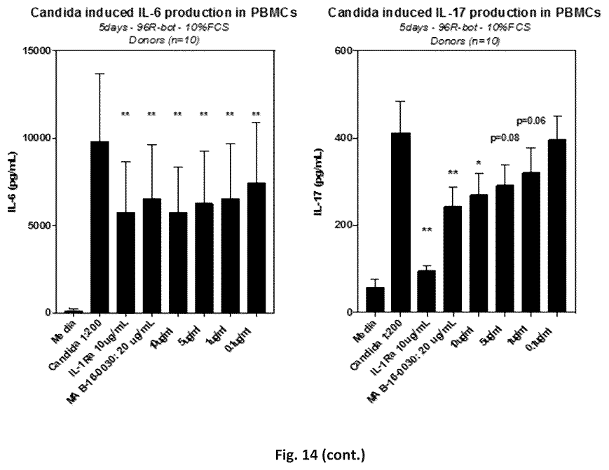

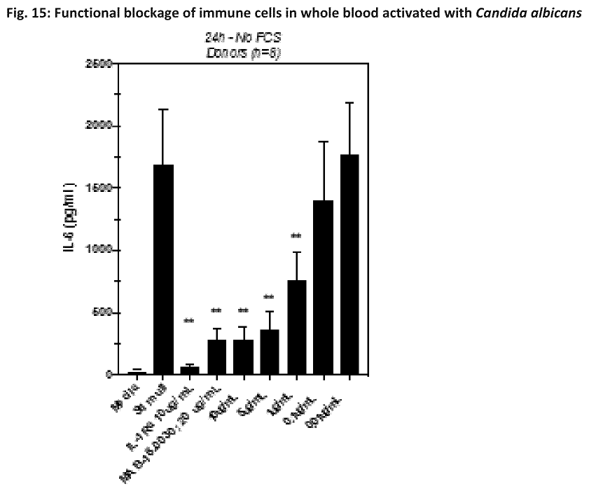

Importantly, the antibodies according to the invention do not affect the viability of immune cells. For example, they do not affect the viability of human peripheral blood mononuclear cells (PBMCs)) and they do not induce IL-6 release in PBMCs (cf. FIG. 13).

The antibodies according to the invention, do not only inhibit the functional activation of cytokine release in different cell lines as described above, but also in PMBCs or whole blood cells from donors. They inhibit cytokine release mediated by different specific or complex stimuli. For example, they inhibit activation of PBMCs stimulated with LPS, heat-inactivated Candida albicans, IL-12/IL-33 or anti-CD3/CD28 antibodies (cf. FIGS. 14 and 15).

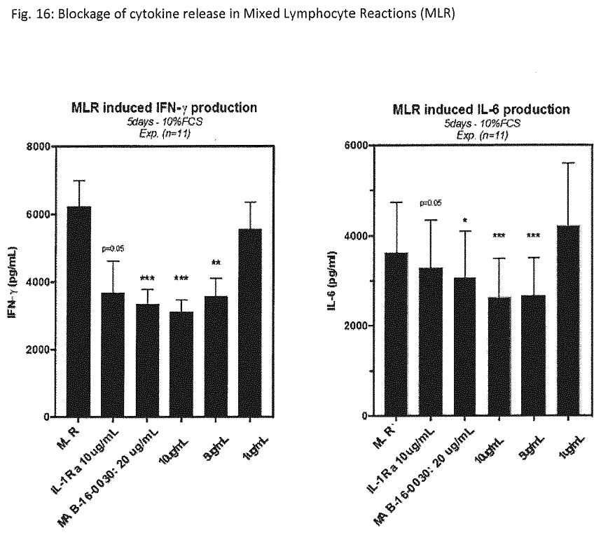

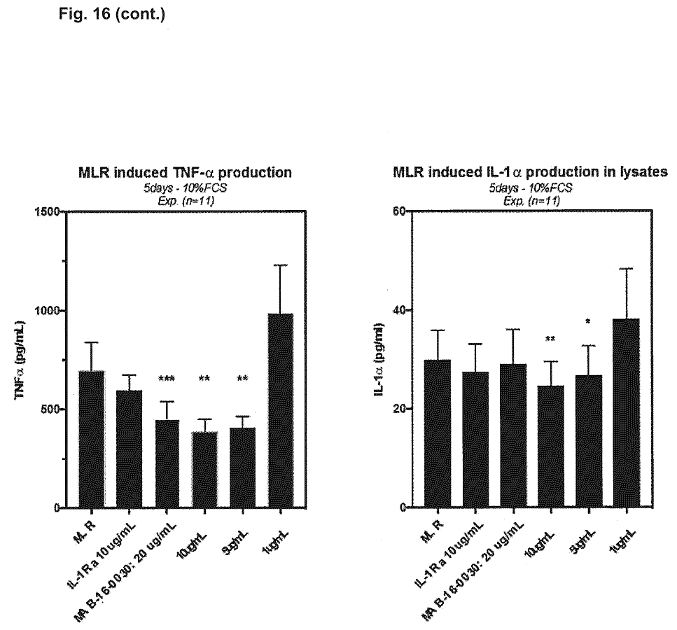

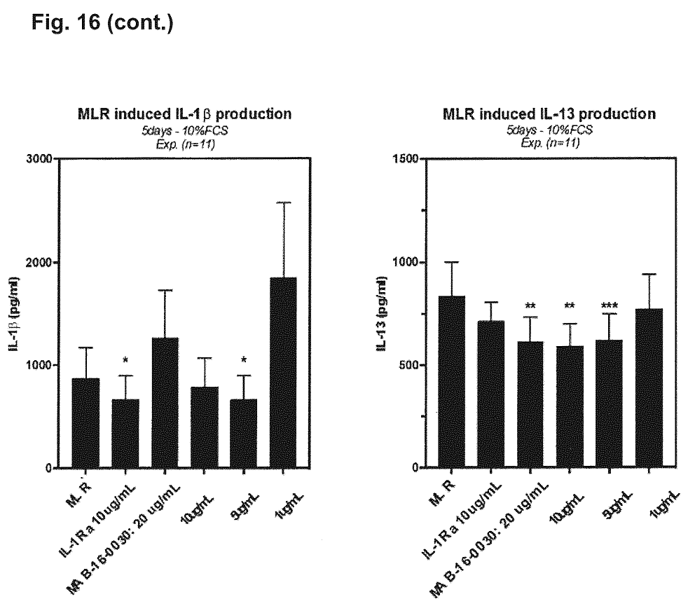

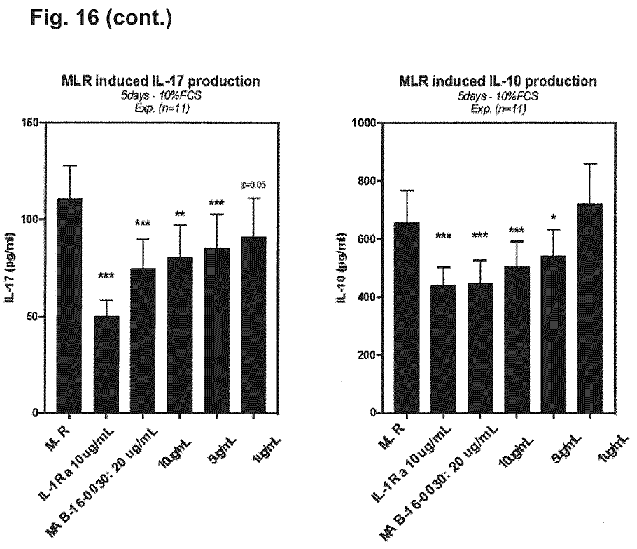

Also, in one embodiment, the humanized anti-IL-1R3 IgG1-LALA antibodies according to the invention are able to inhibit release of IFNg, IL-6, TNF-a, IL-13, IL-17 and IL-10 in mixed lymphocyte reactions (cf. FIG. 16).

Furthermore, the antibodies according to the invention, are especially useful for the treatment of diseases where the dysregulation of the target, IL1R3, is the underlying reason.

Therefore, the present invention further relates to a humanized antibody that specifically binds to IL-1R3 or a fragment or derivative thereof that contains at least a portion of said antibody that is sufficient to confer IL-1R3 binding specificity, for the use in treating a IL-1R3 mediated disease.

The present invention also encompasses a method of treating an IL-1R3 mediated disease in a patient, comprising administering to a patient a pharmaceutically effective amount of the antibody, or derivative or fragment thereof according to the invention.

The invention also relates to a pharmaceutical composition comprising a pharmaceutically acceptable carrier and a therapeutically effective amount of the humanized antibody that specifically binds to the IL-1R3 or a fragment or derivative thereof that contains at least a portion of said antibody that is sufficient to confer IL-1R3 binding specificity, according to the invention.

As used herein, “pharmaceutical carrier” includes any and all solvents, dispersion media, coatings, antibacterial and antifungal agents, isotonic and absorption delaying agents, and the like that are physiologically compatible. Preferably, the carrier is suitable for intravenous, intramuscular, subcutaneous, parenteral, spinal or epidermal administration (e.g. by injection or infusion).

A composition of the present invention can be administered by a variety of methods known in the art. As will be appreciated by the skilled artisan, the route and/or mode of administration will vary depending upon the desired results. To administer a compound of the invention by certain routes of administration, it may be necessary to coat the compound with, or co-administer the compound with, a material to prevent its inactivation. For example, the compound may be administered to a subject in an appropriate carrier, for example, liposomes, or a diluent. Pharmaceutically acceptable diluents include saline and aqueous buffer solutions. Pharmaceutical carriers include sterile aqueous solutions or dispersions and sterile powders for the extemporaneous preparation of sterile injectable solutions or dispersion. The use of such media and agents for pharmaceutically active substances is known in the art.

The phrases “parenteral administration” and “administered parenterally” as used herein means modes of administration other than enteral and topical administration, usually by injection, and includes, without limitation, intravenous, intramuscular, intra-arterial, intrathecal, intracapsular, intraorbital, intracardiac, intradermal, intraperitoneal, transtracheal, subcutaneous, subcuticular, intra-articular, subcapsular, subarachnoid, intraspinal, epidural and intrasternal injection and infusion.

These compositions may also contain adjuvants such as preservatives, wetting agents, emulsifying agents and dispersing agents. Prevention of presence of microorganisms may be ensured both by sterilization procedures, supra, and by the inclusion of various antibacterial and antifungal agents, for example, paraben, chlorobutanol, phenol, sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin. Regardless of the route of administration selected, the compounds of the present invention, which may be used in a suitable hydrated form, and/or the pharmaceutical compositions of the present invention, are formulated into pharmaceutically acceptable dosage forms by conventional methods known to those of skill in the art. Actual dosage levels of the active ingredients in the pharmaceutical compositions of the present invention may be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the activity of the particular compositions of the present invention employed, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts.

One aspect of the invention is a pharmaceutical composition according to the invention for use in the treatment of cancer, as defined in this application.

Another aspect of the invention is a method of treating an IL-1R3 mediated disease in a patient, comprising administering to a patient the pharmaceutical composition according to the invention.

Such IL-1R3 mediated diseases may include cancer. It is described in the literature that a negative prognosis and progression in cancer is associated with increased levels of IL-1α, IL-1β, IL-33, IL-36 and/or increased expression of IL-1R3.

The term “cancer” as used herein may be, for example, lung cancer, non-small cell lung (NSCL) cancer, bronchioloalviolar cell lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, gastric cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, cancer of the bladder, cancer of the kidney or ureter, renal cell carcinoma, carcinoma of the renal pelvis, mesothelioma, hepatocellular cancer, biliary cancer, neoplasms of the central nervous system (CNS), spinal axis tumors, brain stem glioma, glioblastoma multiforme, astrocytomas, schwanomas, ependymonas, medulloblastomas, meningiomas, squamous cell carcinomas, pituitary adenoma, lymphoma, lymphocytic leukemia, including refractory versions of any of the above cancers, or a combination of one or more of the above cancers. Preferably such cancer is a leukemia, breast cancer, colon cancer, lung cancer, or pancreatic cancer. Most preferably the cancer is leukemia.

In one embodiment, the IL-1R3 mediated disease is selected from the group consisting of leukemia, breast cancer, colon cancer, lung cancer, pancreatic cancer, liver cancer, non-small-cell-lung cancer, colorectal cancer, stomach cancer, gastric cancer, estrogen-receptor-positive breast cancer, head and neck squamous cell carcinoma, Mesothelioma, Gall bladder cancer, ovarian cancer, bladder cancer, prostate cancer, Thyroid cancer, Hodgkin disease, MALT lymphoma, salivary gland cancer, or melanoma.

Some tumors are caused or promoted by tumor micro-environment cells secreting inflammatory cytokines such as IL-1α, IL-1β, IL-33, IL-36. In some instances, expression of such cytokines results in the formation of tumor resistance. A concomitant use of cytokine inhibitors and anti-cancer compounds significantly improves the response rate of such treatments or can break tumor resistance. This is provided by the present invention because a broad spectrum of the inhibition of cytokine induced signaling is achieved. Such an activity in cancer indications is not achieved through a direct depletion activity of cancer cells but the inhibition of the cancer associated inflammation by modulating IL1R3 signaling pathways. The antibodies of the present invention provide a very much preferred activity profile because they enable an effective inhibition of cancer associated chronic inflammation and, at the same time, avoid undesired side effects, because they do not exhibit antibody effector function and thus do not affect the viability of targeted cells that express IL-1R3.

Therefore, in one embodiment of the present invention, the antibody or pharmaceutical composition is used for the treatment of patients, wherein the patients comprise a tumor, such as a solid tumor, and show insufficient response or tumor resistance to cytotoxic, cytostatic or targeted/immunotherapy.

In one aspect of the invention, the humanized antibody and/or the pharmaceutical composition according to invention, is meant for use in the treatment of cancer in combination with one or more cytotoxic, cytostatic or targeted anti-cancer compounds. The use of cytokine inhibitors and cytotoxic, cytostatic or targeted anti-cancer compounds significantly improves the response rate of such treatments or can break tumor resistance.

In such aspects of the invention, the cancer is preferably selected from the group consisting of pancreatic cancer, liver cancer, lung cancer (associated with inflammation caused by asbestos, infections, smoking, silica), non-small-cell-lung cancer, colorectal cancer/colitis-associated cancer (associated with Inflammatory bowel disease), stomach cancer, gastric cancer, chronic gastritis associated gastric cancer, estrogen-receptor-positive breast cancer, head and neck squamous cell carcinoma, Mesothelioma, Gall bladder cancer (Gall bladder stone-associated chronic cholecystitis associated), ovarian cancer, bladder cancer, prostate cancer, E. coli-infection associated prostate cancer, Thyroid cancer, Hodgkin disease, MALT lymphoma, salivary gland cancer, melanoma, endometriosis associated endometrial carcinoma, Barrett's esophagitis associated Esophageal cancer.

In one aspect of the method of this invention, the IL1R3 antibody or pharmaceutical composition according to the invention is administered simultaneously with one or more cytotoxic, cytostatic or targeted anti-cancer agents. In another aspect, the antibody or pharmaceutical composition is administered sequentially with one or more cytotoxic, cytostatic or targeted anti-cancer agents.

In the latter case, it is preferred that the antibody is administered after treatment with one or more cytotoxic, cytostatic or targeted anti-cancer agents.

The cytotoxic or cytostatic agents according to the invention can be taxanes, anthracyclins, alkylating agents, Histone Deacetylase Inhibitors, Topoisomerase inhibitors, kinase inhibitors, nucleotide analogs, peptide antibiotics, platinum-based agents and checkpoint inhibitors.

Preferably the targeted anti-cancer agents selected from one of the following, or combinations thereof: anti-EGFR compounds such as cetuximab, gefitinib, erlotinib, lapatinib, panitumumab, and anti-HER2 compounds such as trastuzumab, ado-trastuzumab emtansine, pertuzumab.

It is further preferred that the targeted anti-cancer agents are checkpoint inhibitors. These can be but are not limited to: anti-PD1 compounds such as pembrolizumab, and nivolumab, and anti-PDL1 compounds such as atezolizumab, Avelumab, and Durvalumab, and anti-CTLA-4 compounds such as Ipilimumab and Tremelimumab.

### Examples

The following examples are used in conjunction with the figures and tables to illustrate the invention.

### Example 1: Determination of P013_03 (Human IL-1R3) Specific Antibodies in B-cell Supernatants

### Assay Principle:

NUNC Maxisorp 384well microtiter plates are coated with P013_03. After a blocking process, specific antibodies from B-cell supernatants bind to the antigen and are then detected by a POD-labeled antibody. Samples are tested 1:2 diluted.

### Materials:

Plates: 384well NUNC Maxisorp plates; Cat. No. 464718
Proteins: P013-03 (Conc. 1.5 mg/ml; Assay Conc. 0.5 μg/ml)
Standard Ab: P013-02 (Conc. 1 mg/ml; Start Assay Conc. 2 μg/ml)
Detection Ab: Anti-rabbit IgG, peroxidase-linked species-specific whole antibody (from donkey) (ECL); GE; Cat. No. NA9340; assay dilution: 1:5000
PBS: Buffers in a Box, Premixed PBS Buffer, 10×; Roche Applied Sciences; Cat. No. 11666789001
BSA: Bovine Serum Albumin Fraction V from bovine serum; Roche Applied Sciences; Cat. No. 10735086001
Tween 20: Tween 20; Carl Roth; Cat. No. 9127.2
TMB: TMB Solution; Life Technologies; Cat. No. SB02
HCl: 1M Titripur Hydrochloric Acid; Merck; Cat. No. 1090571000
ELISA Buffer: PBS, 0.5% BSA, 0.05% Tween
Wash Buffer: PBS, 0.1% Tween
Block Buffer: PBS, 2% BSA, 0.05% Tween
Samples: 1:2 dilution in Elisa Buffer

### Procedure:

1. Add 12.5 μL P013-03 (0.5 μg/ml) in PBS to a 384well NUNC Maxisorp plate and incubate for 1 h at RT.
2. Wash 3× with 90 μl Wash Buffer.
3. Add 90 μL Blocking buffer to each well and incubate for 1 h at RT.
4. Wash 3× with Wash Buffer.
5. Add 12.5 μL Standard Antibody in 1:2 dilutions or sample 1:2 diluted in Elisa Buffer and incubate for 1 h at RT.
6. Wash 3× with Wash Buffer.
7. Add 12.5 μL 1:5000 POD-Antibody in Elisa Buffer and incubate for 1 h at RT.
8. Wash 6× with Wash Buffer.
9. Add 15 μL TMB.
10. Add 15 μL HCl after sufficient development.
11. Read absorbance at 450 nm/620 nm.

### Example 2: Identification of P013-Specific Antibodies Inhibiting P013-Receptor by Luciferase Reporter Experiment

### Assay Principle:

293T/17-FR cells, which express a NF-kB-RE firefly luciferase reporter, are seeded into Poly-D-Lysin-Cell culture plates. After stimulation of P013 the 293T/17-FR lysate is tested for activated NF-kB using the Steady-Glo Luciferase Assay Kit. Supernatants with functional antibodies bind to P113 and inhibit the NF-kB activation, which is shown in low signal. Samples are tested 1:2 diluted in P113 solution.

### Materials:

Plates: Cell plate: 384well PDL Costar Cell Culture plate; Cat. No. 3844
Assay plate: 384well lumitrac white-plate; Corning; Cat. No. 3572
Cells: 293T/17-FR; assay conc. 250.000cells/ml
   The higher the cell-passage the lower the signal!
Proteins: P013_05 (Conc. 0.03 mg/ml; Assay Conc. 115 μg/ml; Working Conc. 230 μg/ml)
Standard Ab: P013_06 (Conc. 0.2 mg/ml; Start Working Conc. 6 μg/ml)
Kit: Steady-Glo Luciferase Assay System; Promega; Cat. No. E2510
Cell-Medium: DMEM Medium; PAN Biotech; Cat. No. P04-04510
FCS: Fetal Bovine Serum, HyClone; Thermo; Cat. No. St30070.03 293T/17-FR Medium: DMEM Medium, 10% FCS, (+20 μg/ml Hygromycin-B, just for cultivation)
Conditioned B-cell Medium (MAB Discovery)
Samples: 1:2 dilution with P013_05 in DMEM-Medium+10% FCS

### Procedure:

1. Cell Culture Procedure:
   Split confluent 293T/17-FR cells every Monday (seed out: 5×106 cells/T175 flask) and Friday (seed out: 3×106 cells/T175 flask) using trypsin/EDTA (incubate just for 30 sec at RT).
2. Seed cells (0.25×106 cells/ml) in 25 μl DMEM +10% FCS to a 384-well PDL-plate (Corning cat #3844) and incubate over night at 37° C. and 5% CO2.
3. Aspirate media and add 12.5 μl Sample or P013_06 in 1:3 dilution in Conditioned Medium or just Conditioned Medium and incubate for 30 min at 37° C. and 5% CO2 (program: 3 Aspiration and Sample transfer)
4. Add 12.5 μl P013_05 in DMEM+10% FCS and incubate for 5 hours at 37° C. and 5% CO2 (program: 4_Add P013_05).
5. Equilibrate cultured cells to RT for 10 min.
6. Add 25 μl Steady-Glo Reagent and mix several times with pipette (program: 6_Steady Glo)
7. Wait 5 minutes before transfer 45 μl supernatant to a 384-well lumitrac white plate (Corning Cat #3572) (program: 7_Transfer 45 ul)
8. Measure luminescence in Tecan Reader: Integration Time: 0.5 sec

### Example 3: Determination of hulL1RaP, msIL1Rap, CD134 and CD137 Specific Antibodies in B-Cell Supernatants

### Assay Principle:

NUNC Maxisorp 384well microtiter plates are coated with the target protein. After a blocking process, specific antibodies from B-cell supernatants bind to the targets and are then detected by a POD-labeled antibody.

### Materials:

Plates: 384well NUNC Maxisorp plates; Cat. No. 464718
Proteins: Cleaved hulL1RaP (P026_12; conc. 0.96 mg/ml; assay conc. 0.25 μg/mL)
   Cleaved mulL1RaP (P026_13; conc. 0.93 mg/ml; assay conc. 0.25 μg/mL)
   Cleaved CD134 (P026_14; conc. 0.51 mg/ml; assay conc. 0.25 μg/mL)
   Cleaved CD137 (P026_15; Batch2; conc. 1.1 mg/ml; assay conc. 1 ƒg/mL)
Standard Abs: Human IL-1 RAcP/IL-1 R3 Antibody (PO13_6/P026_08; conc. 0.2 mg/ml or 0.399 mg/ml; start assay conc. 2 μg/ml; used for hulL1RaP and msIL1Rap)
   MAB-14-0283 (conc. 0.6 mg/ml; start assay conc. 2 μg/mL; used for CD134)
   MAB-14-0285 (conc. 1 mg/ml; start assay conc. 2 μg/ml; used for CD137)
Detection Ab: Samples: Anti-rabbit IgG, peroxidase-linked species-specific Fab₂ Fragment (from donkey) (ECL); GE; Cat. No. NA9340; assay dilution: 1:5000 in ELISA buffer
   For MAB-14-0283 and MAB-0285: Anti-human IgG peroxidase-linked species-specific Fab₂ Fragment (from goat) (HRP); AbD Serotec; Cat. No. STAR126P; assay dilution: 1:5000 in ELISA buffer
   For hulL1RaP and msIL1RAP: Peroxidase-conjugated AffiniPure Donkey Anti-Goat IgG (H+L); Jackson Immuno Research; Cat. No. 705-035-003; assay dilution: 1:5000 in ELISA Buffer
PBS: Buffers in a Box, Premixed PBS Buffer, 10×; Roche Applied Sciences; Cat. No. 11666789001
BSA: Bovine Serum Albumin Fraction V from bovine serum; Roche Applied Sciences; Cat. No. 10735086001
Tween 20: Tween 20; Carl Roth; Cat. No. 9127.2
TMB: TMB Solution; Invitrogen; Cat. No. SB02
HCl: 1M Titripur Hydrochloric Acid; Merck; Cat. No. 1090571000
ELISA Buffer: PBS, 0.5% BSA, 0.05% Tween
Wash Buffer: PBS, 0.1% Tween
Blocking Buffer: PBS, 2% BSA, 0.05% Tween
Samples: 1:4 dilution in ELISA Buffer

### Procedure:

1. Add 12.5 μl 0.25 μg/ml or 1 μg/mL protein diluted in PBS to a 384 well NUNC Maxisorp plate and incubate for 1 h at RT.
2. Wash 3× with 90 UL Wash Buffer.
3. Add 90 μL Blocking buffer to each well and incubate for 1 h at RT.
4. Wash 3× with 90 ul Wash Buffer. Plates can be stored in a dry state for up to 6 weeks at −20° C. sealed with an aluminum foil.
5. Add 12.5 μl Standard Antibody in 1:2 dilution steps or sample (diluted in ELISA Buffer) and incubate for 1 h at RT.
6. Wash 3× with 90 μL Wash Buffer.
7. Add 12.5 μL 1:5000 POD-Antibody in Elisa Buffer and incubate for 1 h at RT.
8. Wash 6× with 90 μL Wash Buffer.
9. Add 15 μL TMB.
10. Add 15 μL HCl after 15 min development.
11. Read absorbance at 450 nm/620 nm

### Example 4: Inhibition of NFκB-Expression of A549-NFκB-RE-Luc Stable Transfected Cells After Stimulation with IL-1 (α/β)

### Assay Principle:

A549-NFκB-RE-Luc stable transfected cells (Signosis) are pipetted to a 384-well plate and incubated overnight. On day 2 anti-IL1R3 antibodies are allowed to bind to A549-NFkB-RE-Luc stable transfected cells, which are then stimulated by addition of IL-1 (α or β). This results in transcription of the luciferase gene due to NFκB signaling pathway activation and can be measured by cell lysis and addition of luciferin.

It is tested whether antibodies can inhibit the activation of NFkB pathway and therefore lower the luminescence signal.

### Materials:

Plates: 384-well Low Flange White Flat Bottom Polystyrene TC-Treated Microplates Sterile; Corning; Cat. No. 3570
Proteins: IL-1 α (P026_09); Recombinant Human IL-1alpha/IL-1F1; 10 μg/mL; R&D Systems; Cat. No. 200-LA-002
   IL-1 β (P026_10); Recombinant Human IL-1beta/IL-1F2; 25 μg/ml; R&D Systems; Cat. No. 200-LB-005
Standard Ab: MAB-15-0115; MAB Discovery GmbH; 2.51 mg/ml; working conc. 10 μg/ml
Cells: A549-NFκB-RE-Luc stable transfected cells; Signosis; Cat. No. SL-0014
Medium: DMEM; PAN; Cat. No. P04-04510
FCS: Fetal Bovine Serum South Africa Low IgG; PAN; Cat. No. 1552-P120909
Pen/Strep: 10,000 U Penicillin/ml; 10 mg Streptomycin/ml; PAN Biotech; Cat. No. P06-07100
Detaching Agent: Trypsin-EDTA 1×; PAN; Cat. No. P10-023100 (4 mL for T175/2 mL for T75; ˜8 min 37° C.)
Cell-Medium: DMEM, 10% FCS, 1% Pen/Strep
Detection Kit: Steady-Glo™ Luciferase Assay System; Promega; Cat. No. E2510

### Procedure:

1. Cultivate A549-NFκB-RE-Luc stable transfected cells (1.7E+04 cells/cm² for 3 days; 2,28E+04 cells/cm² for 2 days) in Cell-Medium. Do not go beyond 10 passages!
2. Plate out 40,000 A549-NFκB-RE-Luc stable transfected cells in 25 μl medium per well (conc.=1.6×10⁶ cells/mL) to a white cell-culture treated 384 well plate with flat bottom.

Incubate over night at 37° C./5% CO₂.
3. Aspirate medium from plate and add 10 μl sample or standard in medium to plate using CyBio pipetting roboter (Program: “Medium removal and sample transfer” in folder P026/NFκB). Incubate for 1 h at 37° C./5% CO₂.
4. Add 10 μL IL-1 (α or β) in medium to plate using CyBio pipetting roboter (Program: “Transfer from reservoir” in folder P026/NFκB) (working conc.: 0.2 ng/ml; assay conc.: 0.1 ng/ml) and incubate 5 h at 37° C./5% CO₂.

Before performing step 4, dissolve Steady-Glo substrate in Steady-Glo buffer according to Steady-Glo protocol and equilibrate this solution and the assay plate to RT.
5. Add 20 μl Steady-Glo mix, mix thoroughly to guarantee proper cell lysis. Incubate at RT, 10 min.
6. Determine the relative luminescence units of each well, using a microplate reader set to 500 ms integration time (program: Lumineszenz-384).

### Example 5: IL-6 Secretion of A549 Cells After Stimulation with IL-1 (α/β)

### Assay Principle:

A549 cells are pipetted to a 384-well plate and incubated with anti-IL1R3 antibodies. Afterwards, the cells are stimulated with IL-1 (α or β) and secrete IL-6 into the assay medium. The amount of IL-6 is measured by an IL-6 ELISA.

### Materials:

Assay Kit: DuoSet ELISA Human IL-6; Cat. No. DY206-05 (R&D Systems); DuoSet is consisting out of Human IL-6 Capture Antibody (part 840113), Human IL-6 Detection Antibody (part 840114), Human IL-6 (part 840115) and Streptavidin-HRP (part 8939755)
Plates: 384-well clear cell culture treated plates; Corning; Cat. No. 3701 384-well Maxisorp plates; NUNC; Cat. No. 464718
PP-Plate: 120 μL 384 Deep Well “Diamond” Plate, Clear; Axygen (Corning); Cat. No.P-384-120 SQ-C
Proteins: IL-1 α; Recombinant Human IL-1alpha/IL-1F1; R&D Systems; Cat. No. 200-LA-002
   IL-1 β; Recombinant Human IL-1beta/IL-1F2; R&D Systems; Cat. No. 200-LB-005 rhIL1-ra/IL-1F3; R&D Systems; Cat. No. 280-RA-010
Standard Abs: Human IL-1RAcP/IL-1R3 antibody; R&D Systems; Cat. No. AF676 or AF676-SP
Medium: DMEM; PAN; Cat. No. P04-04510
FCS: Fetal Bovine Serum South Africa Low IgG; PAN; Cat. no. 1552-P120909
Pen/Strep: 10,000 U Penicillin/ml; 10 mg Streptomycin/ml; PAN Biotech; Cat. no. P06-07100
PBS: Buffers in a Box, Premixed PBS Buffer, 10×; Roche Applied Sciences; Cat. No. 11666789001
BSA: Bovine Serum Albumin Fraction V from bovine serum; Roche Applied Sciences; Cat. No. 10735086001
Tween 20: Tween 20; Carl Roth; Cat. No. 9127.2
TMB: TMB Solution; Invitrogen; Cat. No. SB02
HCl: 1 M Titripur Hydrochloric Acid; Merck; Cat. No. 1090571000
ELISA Buffer: PBS, 0.5% BSA, 0.05% Tween
Wash Buffer: PBS, 0.1% Tween
Block Buffer: PBS, 2% BSA, 0.05% Tween
Cell-Medium: DMEM, 10% FCS, 1% Pen/Strep

### Procedure:

### Cell Stimulation

1. Plate out 6,000 A549 cells in 25 μL medium per well (conc.=2.4×10⁵ cells/mL) to a cell culture plate. Incubate over night at 37° C./5% CO₂.
2. Aspirate medium from plate and add 12.5 μl sample or standard in medium to plate. Incubate for 3 h at 37° C./5% CO₂.
3. Add 12.5 μL IL-1 (α or β) to plate (working conc.: 0.2 ng/ml; assay conc.: 0.1 ng/ml) and incubate 48 h at 37°° C./5% CO₂.
4. Aspirate medium and transfer to the coated and blocked Elisa plate (step 9.). Alternatively, the supernatants can be stored at −80° C. in a PP-Plate for up to one week.

Elisa Plate Preparation
5. Dilute the Capture Antibody to a concentration of 2 μg/mL in PBS. Immediately coat a 384-well Maxisorp plate with 12.5 μl per well of the diluted Capture Antibody. Seal the plate and incubate 1 h at room temperature.
6. Aspirate each well and wash with Wash Buffer, repeating the process two times for a total of three washes. Wash by filling each well with Wash Buffer (90 μL) using an autowasher. Complete removal of liquid at each step is essential for good performance. After the last wash, remove any remaining Wash Buffer by aspirating or by inverting the plate and blotting it against clean paper towels.
7. Block plates by adding 90 μL Blocking Buffer to each well. Incubate at room temperature for a minimum of 1 h.
8. Repeat the aspiration/wash as in step 6. The plates are now ready for sample addition. The coated and blocked plates can be stored at −20° C. in a dry state for up to one month.

Assay Procedure
9. Add 12.5 μL of pure sample or IL-6 standard diluted in ELISA Buffer (EB) per well. Cover and incubate 1h at room temperature.
10. Repeat the aspiration/wash as in step 6 of Elisa Plate Preparation.
11. Add 12.5 L of the Detection Antibody, diluted in EB, to each well. Cover and incubate 1 h at room temperature.
12. Repeat the aspiration/wash as in step 6 of Elisa Plate Preparation.
13. Add 12.5 μL of 1:40 diluted Streptavidin-HRP in Elisa Buffer to each well. Cover the plate and incubate for 20 minutes at room temperature. Avoid placing the plate in direct light.
14. Repeat the aspiration/wash as in step 6 of Elisa Plate Preparation.
15. Add 15 μL of Substrate Solution (TMB) to each well. Incubate for 20 minutes at room temperature.
16. Add 15 μL of Stop Solution (HCl, 1 M) to each well. Gently tap the plate to ensure thorough mixing.
17. Determine the optical density of each well immediately, using a microplate reader set to 450 nm. If wavelength correction is available, set to 540 nm or 570 nm (program: TMB stop 384 Cytokine). Readings made directly at 450 nm without correction may be higher and less accurate.

### Example 6: Determination of Binding Characteristics of hulL1RaP Specific Antibodies by Competition Assay

### Assay Principle:

NUNC Maxisorp 384well microtiter plates are coated with the reference antibody Can04. During this time the His-tagged target protein is pre-incubated with the second antibody to test and an anti-HIS-POD antibody. Afterwards this preincubation-mix is added to the assay plate and after a sufficient development time the absorbance at 450 nm/620 nm is measured.

### Materials:

Plates: 384well NUNC Maxisorp plates; Cat. No. 464718
Coating Ab: Can04 (MAB Discovery GmbH; CEP Ab no. 184; conc. 1 mg/ml; assay conc. 100 ng/ml)
Protein: hulL1RaP-His protein (P026_01; Fusion_1_Chain_A Homodimer hulL1RaP-His tagged; GeneArt; conc. 3 mg/ml; assay conc. 62.5 ng/ml)
Standard Abs: Can04 (see: “Coating Ab”; start working conc. 3 μg/ml)
Negative control: Her2 antibody (Lifespan; Cat. no. LS-C95808/26358; conc. 225 μg/mL; start working conc. 3 μg/ml)
Detection Ab: anti-HIS POD Antibody (Monoclonal Anti-polyHistidine Peroxidase Conjugate; Sigma; Cat. No. A7058; conc. 7.5 mg/ml; assay conc.: 3.33 μg/ml)
PBS: Buffers in a Box, Premixed PBS Buffer, 10×; Roche Applied Sciences; Cat. No. 11666789001
BSA: Bovine Serum Albumin Fraction V from bovine serum; Roche Applied Sciences; Cat. No. 10735086001
Tween 20: Tween 20; Sigma-Aldrich; Cat. No. P1379
TMB: TMB Solution; Merck; Cat. No. CL07
HCl: 1M Titripur Hydrochloric Acid; Merck; Cat. No. 1090571000
ELISA Buffer: PBS, 0.5% BSA, 0.05% Tween
Wash Buffer: PBS, 0.05% Tween
Block Buffer: PBS, 2% BSA, 0.05% Tween

### Procedure:

12. Prepare the Pre-incubation-mix and incubate for 2 h at RT.
   a. Pre-incubation (in 384well plate)
      i. Mix 10 μl Secondary Antibody dilution series (dilution 1:2; start working conc.: 3 μg/ml) in ELISA buffer or BLANK with
      ii. 10 μl His-tagged protein (assay conc. 62.5 ng/ml) and
      iii. 10 μl anti-HIS POD antibody (assay conc.: 3.33 μg/ml) and incubate for 1 h at RT.
13. In the meantime, coat a NUNC Maxisorp plate with 20 μl Coating Antibody (Can04; assay conc. 100 ng/ml) in PBS and incubate for 1 h at RT.
14. Wash 3× with 90 μl Wash Buffer.
15. Block with 90 μl Blocking Buffer for 1 h at RT.
16. Wash 3× with Wash Buffer.
17. Add 20 μl Pre-incubation-mix in ELISA-Buffer for 1 h at RT.
18. Wash 6× with Wash Buffer.
19. Add 25 μL TMB.
20. Add 25 μL HCl after sufficient development.
21. Read absorbance at 450 nm/620 nm.

### Example 7: IL12 Counter screen

### Assay Principle:

The IL12 binding is used as a counterscreen. The HER proteins are tagged with a linker, huFc and His (HER1 does not have a His-Tag) like the IL12 protein. Antibodies which bind to the tag are positive in both assays, whereas antigen specific antibodies just bind to the HER proteins and not to IL12.

### Materials:

Plates: 384well NUNC Maxisorp plates; Cat. No. 464718
Proteins: Recombinant Human IL-12 RB1 Fc Chimera; R&D Systems; Cat. No. 839-B1; assay conc. 0.5 μg/ml
Standard Abs: IL-12Rbeta1 antibody; GeneTex; Cat. No. GTX103917; 1 mg/mL; start assay conc. 500 ng/ml (then 1:2 dilutions)
Detection Ab: Anti-rabbit IgG, peroxidase-linked species-specific Fab₂ Fragment (from donkey) (ECL); GE; Cat. No. NA9340; assay dilution: 1:5000
Samples: Dilution in ELISA buffer is project dependent (for high concentrated IgGs 1:2 dilution is recommended)

### Procedure:

1. Add 12.5 μL 0.5 μg/mL HER protein in PBS to a 384well NUNC Maxisorp plate and incubate for 1 h at RT.
2. Wash 3× with Wash Buffer.
3. Add 90 μL Blocking buffer to each well and incubate for 1 h at RT.
4. Wash 3× with Wash Buffer. Plates can be frozen for several weeks at −20° C. sealed with an aluminum foil.
5. Add 12.5 μL Standard Antibody in 1:2 dilutions or sample diluted in ELISA buffer and incubate for 1 h at RT (the frozen plates should be thawed shortly before sample application).
6. Wash 3× with Wash Buffer.
7. Add 12.5 μL 1:5000 POD-Antibody in Elisa Buffer and incubate for 1 h at RT.
8. Wash 6× with Wash Buffer.
9. Add 15 μL TMB.
10. Add 15 μL HCl after sufficient development (project dependent; IL12 assay not shorter than other assays).
11. Read absorbance at 450 nm/620 nm.

### Example 8: Cell Binding Analysis

A549 and NIH-3T3 cells were cultured in DMEM+10% FCS. HEK-293 cells were cultured in DMEM+15% FCS and SK-MEL-30 in RPMI+10% FBS. Cells were harvested using Accumax (Sigma), washed with PBS and resuspended in stain buffer (BD Pharmingen). Anti-IL-1R3 antibodies were incubated with the cells in stain buffer for 30 minutes at 4° C. at a concentration of 10 μg/ml. For EC50 SK-MEL-30 cell binding analysis, cells were incubated in a 1:2 dilution series starting with 20 μg/ml. Cells were washed with stain buffer and incubated with Alexa-488 labelled goat-anti-human secondary antibody (Dianova) for 30 minutes at 4° C. Cells were washed with stain buffer and resuspended in buffer containing 1:100 diluted DRAQ7 (Abcam) dead cell stain. Cells were analysed using a BD Accuri C6 Sampler flow cytometer. Fitting curve and EC50 calculation was done using Excel (Microsoft) and XLfit (IDBS).

### Example 9: Biochemical Human-IL-1R3 ELISA

Nunc 384-well Maxisorp plates were coated with recombinant Fc-tagged hIL-1R3 (Ser21-Glu359) at a concentration of 0.25 μg/ml in PBS for 60 minutes at room temperature. Plates were washed threetimes with wash buffer (PBS 0.1% Tween) and blocked with PBS, 0.2% BSA, 0.05% Tween for 60 minutes at room temperature. After three washes with wash buffer, antibodies were added in ELISA buffer (PBS, 0.5% BSA, 0.05% Tween) at concentrations ranging from 6 to 0.03 μg/ml (1:3 dilution series) and were incubated for 60 min at room temperature. Plates were washed three times with wash buffer, followed by incubation with anti-human-IgG peroxidase-linked, species specific F(ab)2 Fragment (goat, AbD Serotec) at a dilution of 1:5000 in ELISA Buffer for 60 minutes at room temperature. Plates were washed six times with wash buffer before TMB substrate solution (Invitrogen, 15 μl/well) was added. After 5 minutes of incubation, stop solution (1M HCl, 15 μl/well) was added and absorbance (450 nm/620 nm) measured using a Tecan M1000 plate reader. Fitting curves and EC50 calculation were done using Excel (Microsoft) and XLfit (IDBS).

### Example 10: IL-1α and IL-1β Functional Neutralization Assay

A-549-NFκB-RE-Luc (Signosis) were cultivated in DMEM, 10% FCS, 1% Pen/Strep for 5 days before they were seeded out in 384-well white flat bottom polystyrene tissue-culture-treated microplates (Corning) at a cell density of 40,000 cells/well in 25 μl medium. Cells were incubated over night at 37° C./5% CO₂. Medium was removed by aspiration and monoclonal or polyclonal (goat-anti-human-IL-1R3, AF676, R&D Systems) antibodies added at various concentrations in a volume of 10 μl medium and incubated for 60 minutes at 37° C./5% CO₂. Recombinant human IL-1α or IL-1β (R&D Systems) proteins were added in 10 μl medium to a final concentration of 0.1 ng/ml and plates were incubated for 5 hours at 37° C./5% CO₂. 20 μl Steady-Glo™ (Promega) solution were added to each well, mixed thoroughly and plates were incubated for 10 minutes at room temperature before luminescence was measured using a Tecan M1000 plate reader. Fitting curves and EC50 calculation were done using Excel (Microsoft) and XLfit (IDBS).

### Example 11: IL-1α and IL-1β Functional Neutralization Assay—A-549 IL6-Release Assay

A549 cells were seeded out at a density of 6,000 cells/well in 25 μl medium in 384-well clear cell culture treated plates (Corning) in DMEM, 10% FCS, 1% Pen/Strep. Cells were incubated over night at 37° C./5% CO₂. Medium was removed by aspiration and monoclonal or polyclonal (goat-anti-human-IL-1R3, AF676, R&D Systems) antibodies were added at various concentrations in a volume of 12.5 μl medium and incubated for three hours at 37° C./5% CO₂. Recombinant human IL-1α or IL-1β (R&D Systems) proteins were added in 12.5 μl medium to a final concentration of 0.1 ng/ml and plates were incubated for 48 hours at 37° C./5% CO₂. Secreted human-IL-6 levels in the cell supernatant were measured using the DuoSet human IL-6 ELISA kit (R&D Systems, Cat.No. DY206-05) according to the manufacturer's instructions. Fitting curves and EC50 calculation were done using Excel (Microsoft) and XLfit (IDBS).

### Example 12: IL-33 Functional Neutralization Assay

HEK-Blue™ IL-33 cells (InvivoGen) were cultivated in DMEM, 10% FCS for 5 days before they were seeded out in 384-well clear, flat bottom, cell culture treated microplates (Corning) at a cell density of 25,000 cells/well in 15 μl medium. Various concentrations of monoclonal or polyclonal (goat-anti-human-IL-1R3, AF676, R&D Systems) antibodies were added in a volume of 5 μl medium and plates were incubated for 60 minutes at 37° C./5% CO₂. Recombinant human IL-33 (R&D Systems) protein was added in 5 μl medium to a final concentration of 5 ng/ml and plates were incubated over night at 37° C./5% CO₂. 5 μl cell supernatants were transferred to clear, flat bottom polystyrene NBS™ microplates (Corning) containing 20 μl 2×QUANTI-Blue reagent (InvivoGen). Plates were incubated at 37° C. for 45 minutes and optical density measured at 655 nm using a Tecan M1000 plate reader. Fitting curves and EC50 calculation were done using Excel (Microsoft) and XLfit (IDBS).

### Example 13: IL-36 Functional Neutralization Assay

HEK293/17-IF Cells (MAB Discovery GmbH) were cultivated in DMEM, 10% FCS, 20 μg/ml hygromycin for 5 days before they were seeded out in 384-well white, flat bottom, cell culture treated plates (Corning) at a cell density of 30,000 cells/well in 20 μl medium. Cells were incubated over night at 37° C./5% CO₂. Medium was removed by aspiration and various concentrations of monoclonal or polyclonal (goat-anti-human-IL-1R3, AF676, R&D Systems) antibodies were added in a volume of 10 μl medium. Plates were incubated for 60 minutes at 37° C./5% CO₂. Recombinant human IL-36g (R&D Systems) protein was added in 10 μl medium to a final concentration of 15 ng/ml and plates were incubated for 5 hours at 37° C./5% CO₂. 20 μl Steady-Glo™ (Promega) solution was added to each well, mixed thoroughly and plates were incubated for 10 minutes at room temperature before luminescence was read using a Tecan M1000 plate reader. Fitting curves and EC50 calculation were done using Excel (Microsoft) and XLfit (IDBS).

### Example 14: Neutralization of IL-1α, IL-33 and IL-36α

The functions of anti-IL-1R3 antibodies were tested on three different cell lines with either IL-1αa, IL-33 or IL-36α to determine the impact on the signaling pathways involving the three IL-1 receptors (IL-1R1, -R4 or -R6) dependent upon IL-1R3 for signaling.

The human epithelial lung cell line A549 was stimulated with IL-1α as a model of IL-1 dependent diseases such as auto-inflammatory diseases. The cell line was cultured in T75 flasks (37° C., 5% CO₂) in complete F-12K media (10% FCS, 1% Pen/Strep) and split on average 2 times/week, not exceeding 15 passages before assaying. A549 cells were seeded out (50.000/well) in a 96 flat-bottom plate, rested for 3 hrs before pre-incubating 1 hr with MAB-16-0030 (20 μg/mL-1 μg/mL) or IL-1Ra (10 μg/mL). Cells were then stimulated with recombinant human IL-1α (50 pg/mL, Peprotech) for 24 hrs before harvesting supernatants and assaying for IL-6 production (Duoset ELISA, RnD Systems).

A human mast cell line (HMC-1) was investigated for IL-33-dependent induction of IL-8 production. The cell line was cultured in T75 flasks (37° C., 5% CO₂) in complete Iscove's modified Dulbeccos's medium (IMDM, 10% FCS, 1% Pen/Strep) and split on average 3 times/week, not having a cell density above 2*10⁶/mL nor exceeding 15 passages before assaying. HMC-1 cells were seeded out (30.000/well) in a 96 flat-bottom plate, rested for 3 hrs before pre-incubating 1 hr with MAB-16-0030 (20 μg/mL-1 μg/mL) or IL-1Ra (10 μg/mL). Cells were then stimulated with recombinant human IL-33 (20 ng/ml, RnD systems) for 24 hrs before harvesting supernatants and assaying for IL-8 production (Duoset ELISA, RnD Systems).

The impact on IL-36 signaling was investigated using a human keratinocytic cell line (HaCaT). The cell line was cultured in T75 flasks (37°° C., 5% CO₂) in complete DMEM (10% FCS, 1% Pen/Strep) and split on average 3 times/week not exceeding 15 passages before assaying. HaCaT cells were seeded out (50.000/well) in a 96 flat-bottom plate, rested for 3 hrs before pre-incubating 1 hr with MAB-16-0030 (20 μg/mL-1 μg/mL) or IL-1Ra (10 μg/mL). Cells were then stimulated with recombinant human IL-36α (50 ng/ml, RnD systems) for 24 hrs before harvesting supernatants and assaying for IL-8 production (Duoset ELISA, RnD Systems).

### Example 15: Viability and IL-6 Release of PBMC

The impact of anti-hIL-1R3 antibody MAB-16-0030 on the viability of unstimulated PBMCs (500.000/well) from three healthy donors was tested using a conventional MTT reduction assay. Briefly, PBMCs (200 μL) were incubated with either media alone or MAB-16-0030 (20 μg/mL). After 24 hrs, 3 and 5 days, PBMCs were incubated for 2 hrs with MTT (20 μL), before measuring absorbance at 570 nM on an ELISA reader. Using the known linearity between absorbance and viable cells converting MTT, the number of viable cells was calculated using media alone as the control set to 100%. At the same day of MTT analysis, supernatants from PBMCs incubated under same conditions and from same donors, were harvested and subsequently assayed for IL-6 production (Duoset ELISA, RnD systems) to evaluate any possible stimulatory effect of MAB-16-0030 alone.

### Example 16: Functional Blockage of PBMCs

Freshly isolated PBMCs from healthy donors were used to evaluate the impact of MAB-16-0030 on human cells stimulated with diverse antigens. For all stimuli, the experiments were carried out using 500.000 PBMCs/well, stimulating in a total volume of 200 μL. Cells were seeded out and incubated with either media alone, MAB-16-0030 (20-0.1 μg/mL) or IL-1Ra (10 μg/mL) for 1 hr before stimulation. The following stimuli were used; LPS (10 ng/mL, 24 hrs, RPMI no FCS), anti-human CD3/CD28 (1.25 μg/mL; 0.5 μg/mL (eBioscience) 3 days, RPMI 10% FCS), IL-12/-33 (2 ng/mL; 20 ng/ml (Peprotech;RnD Systems)), 3 days, RPMI 10% FCS) or heat-inactivated Candida albicans (0.5*10⁶/mL, 5 days, RPMI 10% FCS). After stimulation, supernatants were harvested and assayed for cytokine production using Duoset ELISAs (RnD Systems) according to manufactures protocol.

### Example 17: Functional Blockage of Immune Cells in Whole Blood

Heat-inactivated Candida albicans were used to stimulate whole blood. Freshly harvested blood from healthy donors (EDTA tubes) were distributed in micro-centrifuge tubes (250 μL/tube) and pre-incubated with either media alone (RPMI, no FCS), MAB-16-0030 (20-0.1 μg/mL) or IL-1Ra (10 μg/mL) for 1 hr before stimulation with Candida albicans (0.5*10⁶/mL) to a final volume of 1 mL. After 24 hrs incubation (37° C., 5% CO₂), supernatants were harvested and assayed for cytokine production by ELISA (Duoset, RnD Systems).

### Example 18: Mixed Lymphocyte Reactions (MLR)

PBMCs from healthy, non-matching donors were mixed in a 1:1 ratio (250.000/donor) and incubated for 5 days (RPMI, 10% FCS) with either media alone, MAB-16-0030 (20-1 μg/mL) or IL-1Ra (10 μg/mL). Cytokine production were assayed using a Quansys multiplex platform according to manufacturer's protocol.

### FIGURE LEGEND

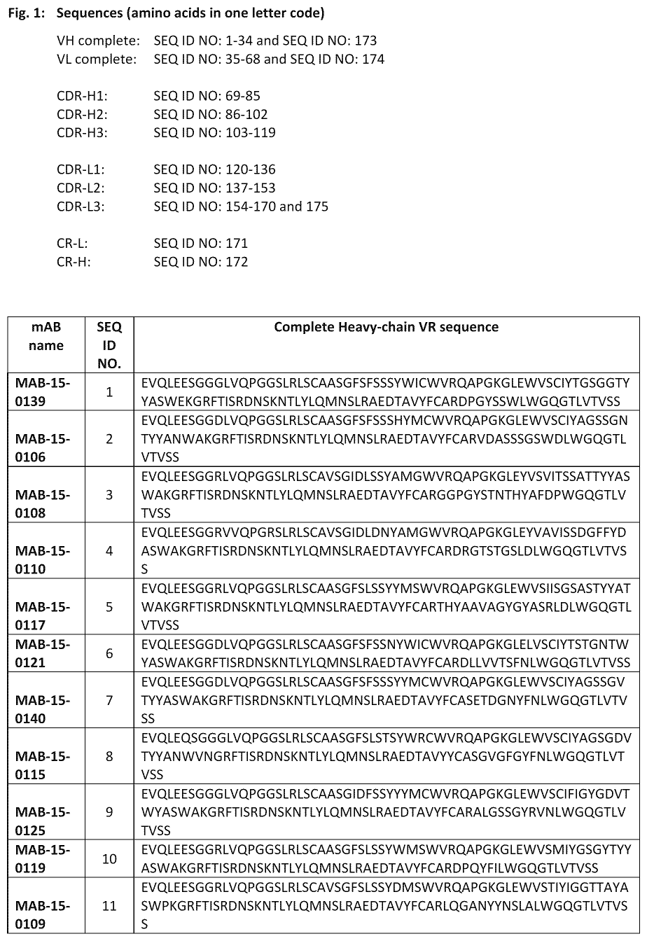

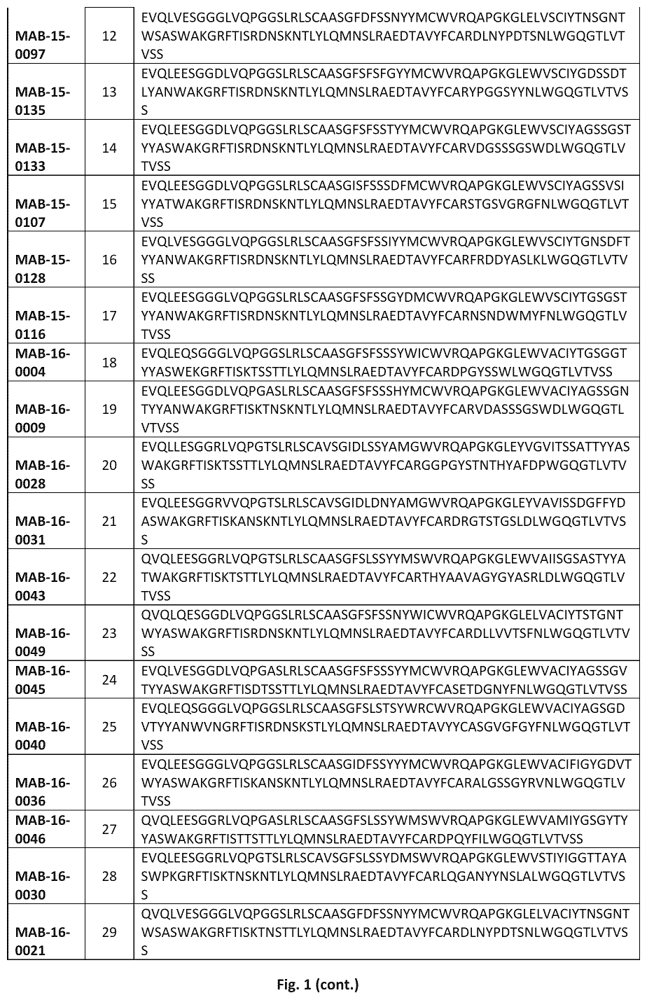

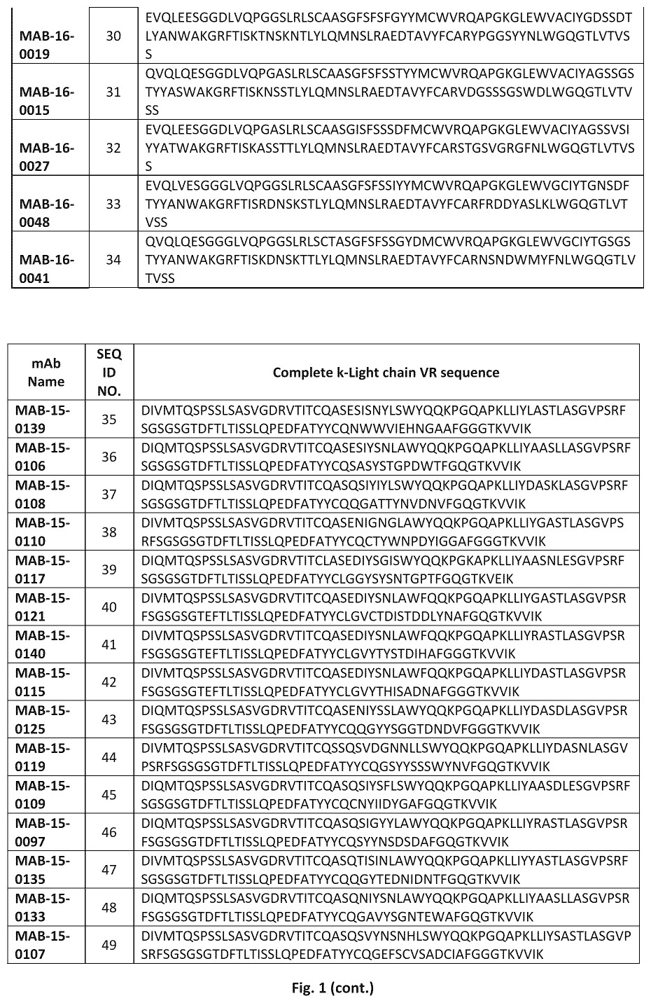

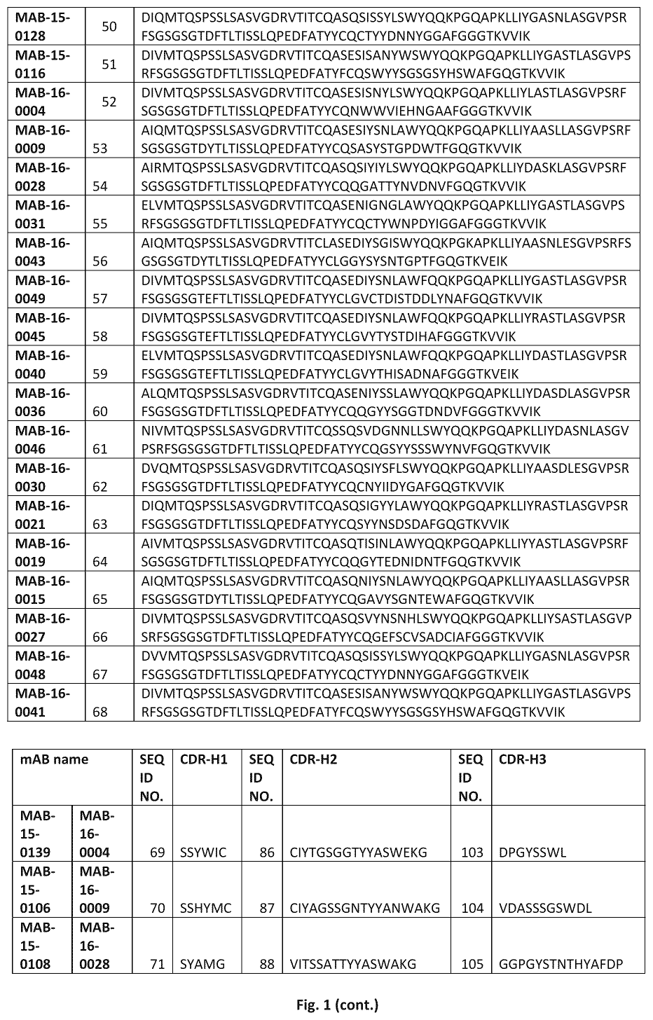

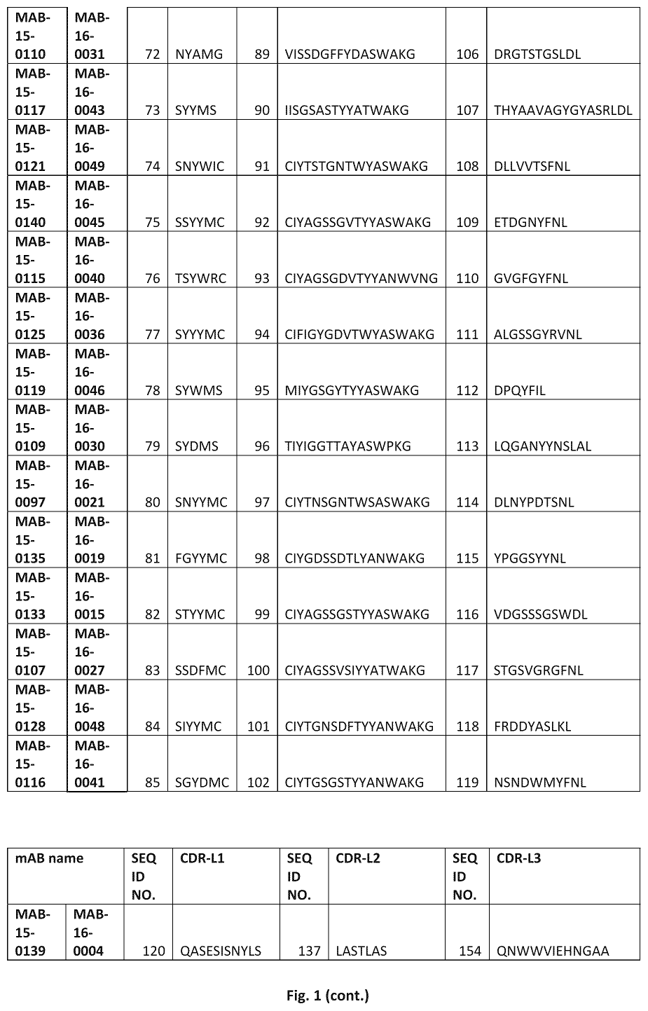

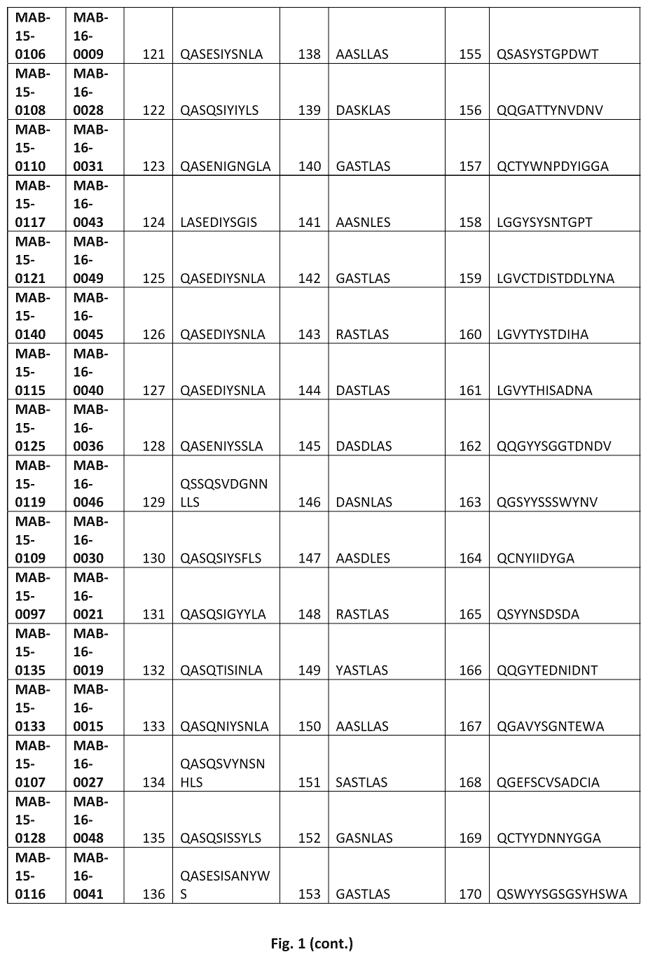

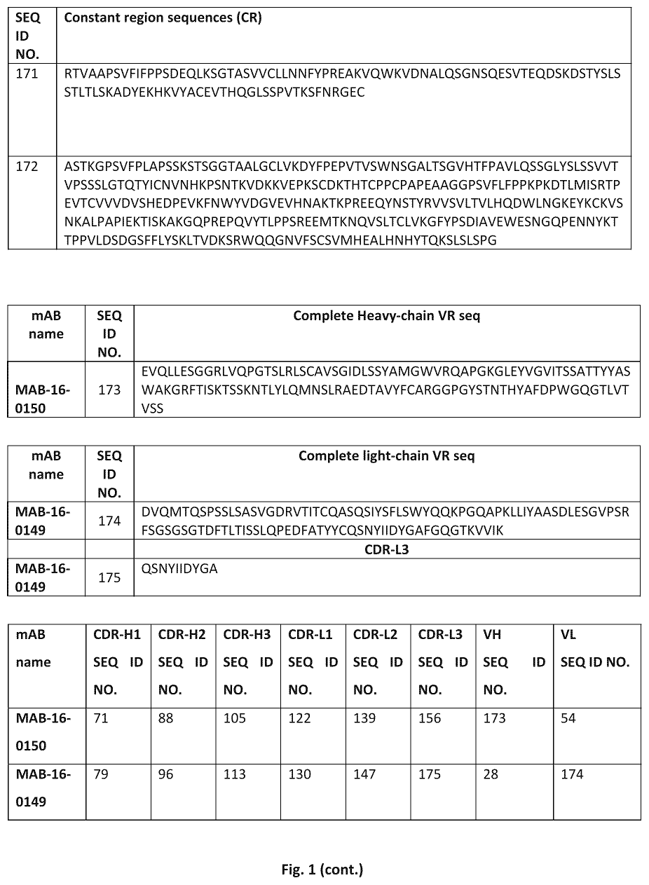

### FIG. 1: Sequences (Amino Acids in One Letter Code)

Complete sequences of Variable Regions (VR):

| | | |
|---|---|---|
| Heavy chain: | VH complete: | SEQ ID NO: 1-34 and |
| | | SEQ ID NO: 173 |
| Light chain: | VL complete: | SEQ ID NO: 35-68 and |
| | | SEQ ID NO: 174 |

Complementary Determining Regions (CDR):

| | | |
|---|---|---|
| Heavy Chain: | CDRH1: | SEQ ID NO: 69-85 |
| | CDRH2: | SEQ ID NO: 86-102 |
| | CDRH3: | SEQ ID NO: 103-119 |
| Light Chain: | CDRL1: | SEQ ID NO: 120-136 |
| | CDRL2: | SEQ ID NO: 137-153 |
| | CDRL3: | SEQ ID NO: 154-170 and 175 |

Constant Regions (CR):

| | | |
|---|---|---|
| Light Chain: | CR-L: | SEQ ID NO: 171 |
| Heavy Chain: | CR-H: | SEQ ID NO: 172 |

In the following Figures, AF676 is a commercial polyclonal antibody preparation purchased from the following link: www.rndsystems.com/products/human-il-1-racp-il-1-r3-antibody_af676.

### FIG. 2: Human IL-1R3 ELISA

A 384 microtiter plate was coated with human Il-1R3 protein representing the human extracellular domain of IL-1R3 (0.5 mg/ml, at least 1 h). After an intensive washing step followed by a blocking step, antibodies were added (12.5 ul per well) and incubated for 1 h at RT. Unbound antibody was washed out intensively. The amount of bound antibody was identified by incubating the microtiter plate with a Peroxidase labelled anti-human detection antibody (1 h at RT). The Peroxidase reaction was initiated by adding TMB and measuring the absorbance at 450 nm/620 nm.

To ensure binding specifically to human IL-1R3, a counter screen using an IL12 protein with identical features was performed in parallel. The set-up of the assay was identical as described above. B-cell supernatants binding to human IL-1R3, but not to IL12 have been regarded as being active.

### FIG. 3: HEK293 Reporter Assay

HEK293T/17-FR cells were stable transfected with the pGL4.32[luc2P/NF-κB-RE/Hygro] vector (Promega) and seeded in 384well PDL Costar Cell Culture plates followed by 30 min incubation with the antibodies. The cells were then stimulated with IL-1β for 5 hours before the NF-kB activity was determined using the Steady-Glo Luciferase Assay Kit (Promega) according to manufacturer's protocol.

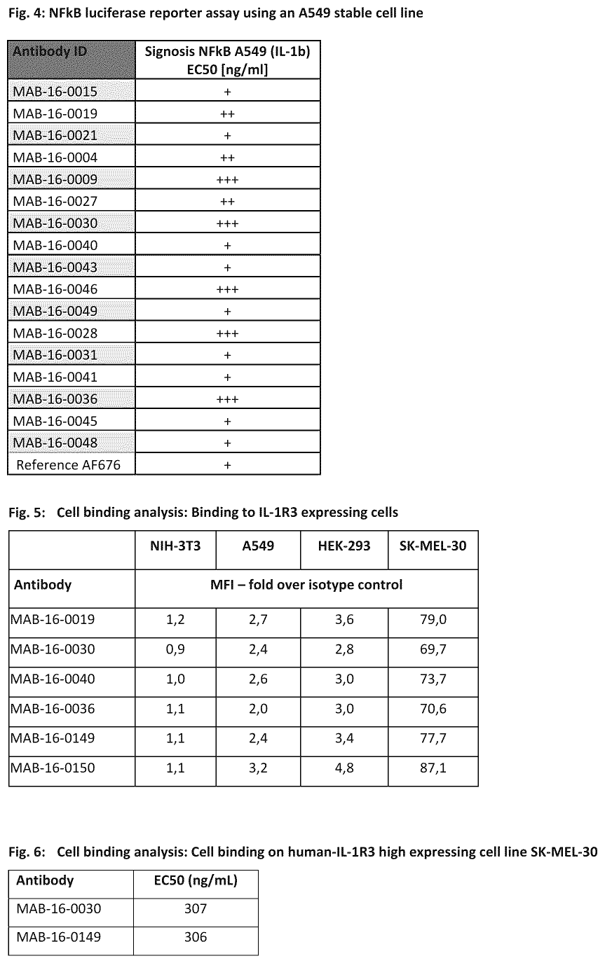

### FIG. 4: NFκB Luciferase Reporter Assay Using an A549 Stable Cell Line

A549-NFkB-RE-Luc stable transfected cells (purchased from Signosis) have been cultivated for 3 days (1,7E+04 cells/cm³). 384-well low flange white flat bottom polystyrene TC-treated microtiter plates (Corning) were filled with 4×10⁴ cells per well. After a cultivation period of 10 h the cells were incubated with the antibodies for 1 h before the cells were stimulated with 10 μl IL-1β for another 5 h. NFkB modulation has been measured by Steady-Glo™ Luciferase Assay System (Promega) determining relative luminescence units of each well in relation to non-stimulated cells.

### FIG. 5: Cell Binding Analysis: Binding to IL-1R3 Expressing Cells

Humanized anti-IL-1R3 IgG1-LALA antibodies were tested for binding to cell lines with different IL-1R3 receptor densities using flow cytometry. Humanized anti-IL-1R3 IgG1-LALA antibodies bind to low-and high-IL-1R3-expressing cell lines. Antibodies do not bind to mouse NIH-3T3 cells. Experiments were carried out according to the method described in Example 8.

### FIG. 6: Cell Binding Analysis: Cell Binding on Human-IL-1R3 High Expressing Cell Line SK-MEL-30

EC50 cell binding values of humanized anti-IL-1R3 IgG1-LALA antibodies were determined by binding to high-IL-1R3-expressing cell line SK-MEL-30 using flow cytometry. Humanized anti-IL-1R3 IgG1-LALA antibodies MAB-16-0030 and MAB-16-0149 show cell binding of 307 and 306 ng/ml, respectively. Experiments were carried out according to the method described in Example 8.

### FIG. 7: Human-IL-1R3 Biochemical ELISA

Binding of humanized anti-IL-1R3 IgG1-LALA antibodies to recombinant human IL-1R3 protein was tested in biochemical ELISA. Exemplified antibodies show EC50 binding values of 16.3 ng/ml and 29.1 ng/ml, respectively. Experiments were carried out according to the method described in Example 9.

### FIG. 8: Inhibition of human IL-1a and IL-1b mediated NfKB signaling in A549-NFkB-RE-Luc cells

Functional neutralization of IL-1a and IL-1b was tested in a cell based gene reporter assay using A549-NFkB-RE-Luc cells stimulated with 0.1 ng/ml IL-1a and IL-1b, respectively. Humanized anti-IL-1R3 IgG1-LALA antibodies show EC50 values superior to that of goat-anti-human-IL1-R3 polyclonal antibody AF676 (R&D Systems). Experiments were carried out according to the method described in Example 10.

### FIG. 9: IL-1a and IL-1βFunctional Neutralization Assay-Inhibition of Human IL-1a and IL-1b Mediated IL-6 Release by A-549 Cells

Neutralization of IL-1a and IL-1b mediated cellular release of IL-6 by humanized anti-IL-1R3 IgG1-LALA antibodies was tested using A-549 cells. EC50 values demonstrate that humanized anti-IL-1R3 IgG1-LALA antibodies are superior to that of goat-anti-human-IL1-R3 polyclonal antibody AF676 (R&D Systems). Experiments were carried out according to the method described in Example 11.

### FIG. 10: IL-33 Functional Neutralization Assay-Inhibition of Human IL-33 mediated NfkB-Signaling in HEK-Blue-IL33™ Cells

Neutralization of IL-33 mediated cell signaling by humanized anti-IL-1R3 IgG1-LALA antibodies was tested using IL-33 stimulated gene reporter HEK-Blue-IL33™ cells (InvivoGen). EC50 values demonstrate that humanized anti-IL-1R3 IgG1-LALA antibodies are superior to that of goat-anti-human-IL1-R3 polyclonal antibody AF676 (R&D Systems). Experiments were carried out according to the method described in Example 12.

### FIG. 11: IL-36 Functional Neutralization Assay-Inhibition of Human IL-36 Mediated NfkB-Signaling in HEK-293/17-IF Cells

Neutralization of IL-36 mediated cell signaling by humanized anti-IL-1R3 IgG1-LALA antibodies was tested using IL-36g stimulated gene reporter HEK-293/17-IF cells. Typical humanized anti-IL-1R3 IgG1-LALA antibodies show EC50 values superior to that of goat-anti-human-IL1-R3 polyclonal antibody AF676 (R&D Systems). Experiments were carried out according to the method described in Example 13.

### FIG. 12: Neutralization of IL-1a, IL-33 and IL-36a Mediated Cellular Cytokine Release

Neutralization of IL-1a, IL-33 and IL-36a mediated cellular cytokine release was tested using specific IL-1a, IL-33 and IL-36a dependent cell systems. The Inhibition of cytokine release by a representative humanized anti-IL-1R3 IgG1-LALA antibody according to the invention (MAB-16-0030) was tested and compared to IL-1Ra. While the antibody according to the invention was able to inhibit cytokine release mediated by all three stimuli, IL-1Ra affected only IL-1a mediated cytokine release. Experiments were carried out according to the method described in Example 14.

### FIG. 13: Viability and IL-6 Release of Ustimulated PBMC Treated with a Humanized Anti-IL-1R3 IgG1-LALA Antibody

Binding of antibodies to immune cells may result in cell depleting and deleterious effects, e.g. by direct induction of apoptotic signaling pathways, stimulation of excessive cytokine release or antibody-dependent cellular cytotoxicity (ADCC). To exclude that humanized anti-IL-1R3 IgG1-LALA antibodies directly affect the viability of PBMCs, the viability of PBMCs of three donors and IL-6 release was investigated after incubation with different concentrations of a representative humanized anti-IL-1R3 IgG1-LALA antibody according to the invention (MAB-16-0030) for 1,3 and 5 days. Neither viability nor IL-6 release was affected by MAB-16-0030. These results support that the humanized anti-IL-1R3 IgG1-LALA antibodies block IL-1R3 function on immune cells without circumstantial cell-depletion and induction of cell-deleterious effects. Experiments were carried out according to the method described in Example 15.

### FIG. 14: Functional Blockage of PBMCs Activated with Different Stimuli

To test whether humanized anti-IL-1R3 IgG1-LALA antibodies inhibit activation of PBMCs stimulated with specific or complex stimuli, PBMCs of 10 donors were stimulated with LPS, heat-inactivated Candida albicans, IL-12/IL-33 or anti-CD3/CD28 antibodies. A representative humanized anti-IL-1R3 IgG1-LALA antibody according to the invention was able to inhibit cytokine release mediated by all tested stimuli. Experiments were carried out according to the method described in Example 16.

### FIG. 15: Functional Blockage of Immune Cells in Whole Blood Activated with Candida albicans

To test whether humanized anti-IL-1R3 IgG1-LALA antibodies inhibit activation of immune cells in whole blood, whole blood of 8 donors was stimulated with heat-inactivated Candida albicans. The representative humanized anti-IL-1R3 IgG1-LALA antibody according to the invention shown in the figure, was able to inhibit Candida induced IL-6 cytokine release. Experiments were carried out according to the method described in Example 17.

### FIG. 16: Blockage of cytokine release in Mixed Lymphocyte Reactions (MLR)

The ability of humanized anti-IL-1R3 IgG1-LALA antibodies to block release of diverse cytokines was tested in mixed lymphocyte reactions (MLR) using PBMCs of healthy, unmatched donors. The representative humanized anti-IL-1R3 IgG1-LALA antibody according to the invention shown in the figure was able to inhibit release of IFNg, IL-6, TNF-a, IL-13, IL-17 and IL-10. Experiments were carried out according to the method described in Example 18.

1-19. (canceled)

20. A method of treating a IL-1R3 mediated disease in a patient suffering therefrom, comprising administering to a patient an antibody that specifically binds to IL-1R3 or a fragment or derivative thereof, the antibody or fragment or derivative thereof comprising:
a) a heavy chain variable region (VH) comprising the complementarity determining regions comprising CDR-H1, CDR-H2, and CDR-H3 wherein the CDR-H1 region comprises an amino acid sequence set forth in SEQ ID NO: 79, wherein the CDR-H2 region comprises an amino acid sequence set forth in SEQ ID NO: 96, and wherein the CDR-H3 region comprises an amino acid sequence set forth in SEQ ID NO: 113; and
b) a light chain variable region (VL) comprising the complementarity determining regions comprising CDR-L1, CDR-L2, and CDR-L3 wherein the CDR-L1 region comprises an amino acid sequence set forth in SEQ ID NO: 130, wherein the CDR-L2 region comprises an amino acid sequence set forth in SEQ ID NO: 147, and wherein the CDR-L3 region comprises an amino acid sequence set forth in SEQ ID NO: 175.

21. The method of claim 20, wherein the heavy chain variable (VH) region comprises an amino acid sequence set forth in SEQ ID NO: 28, and the light chain variable (VL) region comprises an amino acid sequence set forth in SEQ ID NO: 174.

22. The method of claim 20, wherein the antibody or fragment or derivative thereof comprises a human IgG1 Fc region comprising at least amino acid substitutions at L234A and L235A of the human IgG1 Fc region, wherein the numbering of amino acid residues in the Fc region or constant region is according to the EU numbering system.

23. The method of claim 20, wherein the antibody or fragment or derivative thereof comprises a human IgG4 Fc region comprising at least amino acid substitutions at S228P and L235E of the human IgG4 Fc region, wherein the numbering of amino acid residues in the Fc region or constant region is according to the EU numbering system.

24. The method of claim 20, wherein the IL-1R3 mediated disease is cancer.

25. The method of claim 24, wherein the cancer is selected from the group consisting of lung cancer, non-small cell lung (NSCL) cancer, bronchioloalviolar cell lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, gastric cancer, colon cancer, breast cancer, or uterine cancer.

26. The method of claim 24, wherein the cancer is leukemia or lymphoma.

27. The method of claim 24, wherein the antibody, or fragment or derivative thereof is administered in combination with one or more cytotoxic, cytostatic or targeted anti-cancer compounds.

28. A method of treating an IL-1R3 mediated disease in a patient, comprising administering to a patient an antibody that specifically binds to IL-1R3, wherein the antibody comprises:
a) a VH region comprising an amino acid sequence set forth in SEQ ID NO: 28;
b) a VL region comprising an amino acid sequence set forth in SEQ ID NO: 174; and
c) a human IgG1 Fc region comprising at least amino acid substitutions at L234A and L235A of the human IgG1 Fc region, wherein the numbering of amino acid residues in the Fc region or constant region is according to the EU numbering system.

29. The method of claim 28, wherein the IL-1R3 mediated disease is cancer.

30. The method of claim 29, wherein the cancer is selected from the group consisting of lung cancer, non-small cell lung (NSCL) cancer, bronchioloalviolar cell lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, gastric cancer, colon cancer, breast cancer, or uterine cancer.

31. The method of claim 28, wherein the cancer is leukemia or lymphoma.

32. The method of claim 28, wherein the antibody is administered in combination with one or more cytotoxic, cytostatic or targeted anti-cancer compounds.

33. A method of treating an IL-1R3 mediated disease in a patient, comprising administering to a patient an antibody that specifically binds to IL-1R3, the antibody comprising:
a) a VH region comprising an amino acid sequence set forth in SEQ ID NO: 28;
b) a VL region comprising an amino acid sequence set forth in SEQ ID NO: 174; and
c) a human IgG4 Fc region comprising at least amino acid substitutions at S228P and L235E of the human IgG4 Fc region, wherein the numbering of amino acid residues in the Fc region or constant region is according to the EU numbering system.

34. The method of claim 33, wherein the IL-1R3 mediated disease is cancer.

35. The method of claim 34, wherein the cancer is selected from the group consisting of lung cancer, non-small cell lung (NSCL) cancer, bronchioloalviolar cell lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, gastric cancer, colon cancer, breast cancer, or uterine cancer.

36. The method of claim 34, wherein the cancer is leukemia or lymphoma.

37. The method of claim 34, wherein the antibody is administered in combination with one or more cytotoxic, cytostatic or targeted anti-cancer compounds.


SEQUENCE LISTING (175 sequences)

SEQ ID NO: 1 - protein, 118 aa, synthetic construct
  REGION 1..118: note = humanized antibody
EVQLEESGGGLVQPGGSLRLSCAASGFSFSSSYWICWVRQAPGKGLEWVSCIYTGSGGTYYASWEKGRFTISRDNSKNTLYLQMNSLRAEDTAVYFCARDPGYSSWLWGQGTLVTVSS

SEQ ID NO: 2 - protein, 122 aa, synthetic construct
  REGION 1..122: note = humanized antibody
EVQLEESGGDLVQPGGSLRLSCAASGFSFSSSHYMCWVRQAPGKGLEWVSCIYAGSSGNTYYANWAKGRFTISRDNSKNTLYLQMNSLRAEDTAVYFCARVDASSSGSWDLWGQGTLVTVSS

SEQ ID NO: 3 - protein, 123 aa, synthetic construct
  REGION 1..123: note = humanized antibody
EVQLEESGGRLVQPGGSLRLSCAVSGIDLSSYAMGWVRQAPGKGLEYVSVITSSATTYYASWAKGRFTISRDNSKNTLYLQMNSLRAEDTAVYFCARGGPGYSTNTHYAFDPWGQGTLVTVSS

SEQ ID NO: 4 - protein, 119 aa, synthetic construct
  REGION 1..119: note = humanized antibody
EVQLEESGGRVVQPGRSLRLSCAVSGIDLDNYAMGWVRQAPGKGLEYVAVISSDGFFYDASWAKGRFTISRDNSKNTLYLQMNSLRAEDTAVYFCARDRGTSTGSLDLWGQGTLVTVSS

SEQ ID NO: 5 - protein, 125 aa, synthetic construct
  REGION 1..125: note = humanized antibody
EVQLEESGGRLVQPGGSLRLSCAASGFSLSSYYMSWVRQAPGKGLEWVSIISGSASTYYATWAKGRFTISRDNSKNTLYLQMNSLRAEDTAVYFCARTHYAAVAGYGYASRLDLWGQGTLVTVSS

SEQ ID NO: 6 - protein, 120 aa, synthetic construct
  REGION 1..120: note = humanized antibody
EVQLEESGGDLVQPGGSLRLSCAASGFSFSSNYWICWVRQAPGKGLELVSCIYTSTGNTWYASWAKGRFTISRDNSKNTLYLQMNSLRAEDTAVYFCARDLLVVTSFNLWGQGTLVTVSS

SEQ ID NO: 7 - protein, 120 aa, synthetic construct
  REGION 1..120: note = humanized antibody
EVQLEESGGDLVQPGGSLRLSCAASGFSFSSSYYMCWVRQAPGKGLEWVSCIYAGSSGVTYYASWAKGRFTISRDNSKNTLYLQMNSLRAEDTAVYFCASETDGNYFNLWGQGTLVTVSS

SEQ ID NO: 8 - protein, 120 aa, synthetic construct
  REGION 1..120: note = humanized antibody
EVQLEQSGGGLVQPGGSLRLSCAASGFSLSTSYWRCWVRQAPGKGLEWVSCIYAGSGDVTYYANWVNGRFTISRDNSKNTLYLQMNSLRAEDTAVYYCASGVGFGYFNLWGQGTLVTVSS

SEQ ID NO: 9 - protein, 122 aa, synthetic construct
  REGION 1..122: note = humanized antibody
EVQLEESGGGLVQPGGSLRLSCAASGIDFSSYYYMCWVRQAPGKGLEWVSCIFIGYGDVTWYASWAKGRFTISRDNSKNTLYLQMNSLRAEDTAVYFCARALGSSGYRVNLWGQGTLVTVSS

SEQ ID NO: 10 - protein, 115 aa, synthetic construct
  REGION 1..115: note = humanized antibody
EVQLEESGGRLVQPGGSLRLSCAASGFSLSSYWMSWVRQAPGKGLEWVSMIYGSGYTYYASWAKGRFTISRDNSKNTLYLQMNSLRAEDTAVYFCARDPQYFILWGQGTLVTVSS

SEQ ID NO: 11 - protein, 120 aa, synthetic construct
  REGION 1..120: note = humanized antibody
EVQLEESGGRLVQPGGSLRLSCAVSGFSLSSYDMSWVRQAPGKGLEWVSTIYIGGTTAYASWPKGRFTISRDNSKNTLYLQMNSLRAEDTAVYFCARLQGANYYNSLALWGQGTLVTVSS

SEQ ID NO: 12 - protein, 120 aa, synthetic construct
  REGION 1..120: note = humanized antibody
EVQLVESGGGLVQPGGSLRLSCAASGFDFSSNYYMCWVRQAPGKGLELVSCIYTNSGNTWSASWAKGRFTISRDNSKNTLYLQMNSLRAEDTAVYFCARDLNYPDTSNLWGQGTLVTVSS

SEQ ID NO: 13 - protein, 119 aa, synthetic construct
  REGION 1..119: note = humanized antibody
EVQLEESGGDLVQPGGSLRLSCAASGFSFSFGYYMCWVRQAPGKGLEWVSCIYGDSSDTLYANWAKGRFTISRDNSKNTLYLQMNSLRAEDTAVYFCARYPGGSYYNLWGQGTLVTVSS

SEQ ID NO: 14 - protein, 122 aa, synthetic construct
  REGION 1..122: note = humanized antibody
EVQLEESGGDLVQPGGSLRLSCAASGFSFSSTYYMCWVRQAPGKGLEWVSCIYAGSSGSTYYASWAKGRFTISRDNSKNTLYLQMNSLRAEDTAVYFCARVDGSSSGSWDLWGQGTLVTVSS

SEQ ID NO: 15 - protein, 122 aa, synthetic construct
  REGION 1..122: note = humanized antibody
EVQLEESGGDLVQPGGSLRLSCAASGISFSSSDFMCWVRQAPGKGLEWVSCIYAGSSVSIYYATWAKGRFTISRDNSKNTLYLQMNSLRAEDTAVYFCARSTGSVGRGFNLWGQGTLVTVSS

SEQ ID NO: 16 - protein, 121 aa, synthetic construct
  REGION 1..121: note = humanized antibody
EVQLVESGGGLVQPGGSLRLSCAASGFSFSSIYYMCWVRQAPGKGLEWVSCIYTGNSDFTYYANWAKGRFTISRDNSKNTLYLQMNSLRAEDTAVYFCARFRDDYASLKLWGQGTLVTVSS

SEQ ID NO: 17 - protein, 120 aa, synthetic construct
  REGION 1..120: note = humanized antibody
EVQLEESGGGLVQPGGSLRLSCAASGFSFSSGYDMCWVRQAPGKGLEWVSCIYTGSGSTYYANWAKGRFTISRDNSKNTLYLQMNSLRAEDTAVYFCARNSNDWMYFNLWGQGTLVTVSS

SEQ ID NO: 18 - protein, 117 aa, synthetic construct
  REGION 1..117: note = humanized antibody
EVQLEQSGGGLVQPGGSLRLSCAASGFSFSSSYWICWVRQAPGKGLEWVACIYTGSGGTYYASWEKGRFTISKTSSTTLYLQMNSLRAEDTAVYFCARDPGYSSWLWGQGTLVTVSS

SEQ ID NO: 19 - protein, 122 aa, synthetic construct
  REGION 1..122: note = humanized antibody
EVQLEESGGDLVQPGASLRLSCAASGFSFSSSHYMCWVRQAPGKGLEWVACIYAGSSGNTYYANWAKGRFTISKTNSKNTLYLQMNSLRAEDTAVYFCARVDASSSGSWDLWGQGTLVTVSS

SEQ ID NO: 20 - protein, 122 aa, synthetic construct
  REGION 1..122: note = humanized antibody
EVQLLESGGRLVQPGTSLRLSCAVSGIDLSSYAMGWVRQAPGKGLEYVGVITSSATTYYASWAKGRFTISKTSSTTLYLQMNSLRAEDTAVYFCARGGPGYSTNTHYAFDPWGQGTLVTVSS

SEQ ID NO: 21 - protein, 119 aa, synthetic construct
  REGION 1..119: note = humanized antibody
EVQLEESGGRVVQPGTSLRLSCAVSGIDLDNYAMGWVRQAPGKGLEYVAVISSDGFFYDASWAKGRFTISKANSKNTLYLQMNSLRAEDTAVYFCARDRGTSTGSLDLWGQGTLVTVSS

SEQ ID NO: 22 - protein, 123 aa, synthetic construct
  REGION 1..123: note = humanized antibody
QVQLEESGGRLVQPGTSLRLSCAASGFSLSSYYMSWVRQAPGKGLEWVAIISGSASTYYATWAKGRFTISKTSTTLYLQMNSLRAEDTAVYFCARTHYAAVAGYGYASRLDLWGQGTLVTVSS

SEQ ID NO: 23 - protein, 120 aa, synthetic construct
  REGION 1..120: note = humanized antibody
QVQLQESGGDLVQPGGSLRLSCAASGFSFSSNYWICWVRQAPGKGLELVACIYTSTGNTWYASWAKGRFTISRDNSKNTLYLQMNSLRAEDTAVYFCARDLLVVTSFNLWGQGTLVTVSS

SEQ ID NO: 24 - protein, 119 aa, synthetic construct
  REGION 1..119: note = humanized antibody
EVQLVESGGDLVQPGASLRLSCAASGFSFSSSYYMCWVRQAPGKGLEWVACIYAGSSGVTYYASWAKGRFTISDTSSTTLYLQMNSLRAEDTAVYFCASETDGNYFNLWGQGTLVTVSS

SEQ ID NO: 25 - protein, 120 aa, synthetic construct
  REGION 1..120: note = humanized antibody
EVQLEQSGGGLVQPGGSLRLSCAASGFSLSTSYWRCWVRQAPGKGLEWVACIYAGSGDVTYYANWVNGRFTISRDNSKSTLYLQMNSLRAEDTAVYYCASGVGFGYFNLWGQGTLVTVSS

SEQ ID NO: 26 - protein, 122 aa, synthetic construct
  REGION 1..122: note = humanized antibody
EVQLEESGGGLVQPGGSLRLSCAASGIDFSSYYYMCWVRQAPGKGLEWVACIFIGYGDVTWYASWAKGRFTISKANSKNTLYLQMNSLRAEDTAVYFCARALGSSGYRVNLWGQGTLVTVSS

SEQ ID NO: 27 - protein, 113 aa, synthetic construct
  REGION 1..113: note = humanized antibody
QVQLEESGGRLVQPGASLRLSCAASGFSLSSYWMSWVRQAPGKGLEWVAMIYGSGYTYYASWAKGRFTISTTSTTLYLQMNSLRAEDTAVYFCARDPQYFILWGQGTLVTVSS

SEQ ID NO: 28 - protein, 120 aa, synthetic construct
  REGION 1..120: note = humanized antibody
EVQLEESGGRLVQPGTSLRLSCAVSGFSLSSYDMSWVRQAPGKGLEWVSTIYIGGTTAYASWPKGRFTISKTNSKNTLYLQMNSLRAEDTAVYFCARLQGANYYNSLALWGQGTLVTVSS

SEQ ID NO: 29 - protein, 119 aa, synthetic construct
  REGION 1..119: note = humanized antibody
QVQLVESGGGLVQPGGSLRLSCAASGFDFSSNYYMCWVRQAPGKGLELVACIYTNSGNTWSASWAKGRFTISKTNSTTLYLQMNSLRAEDTAVYFCARDLNYPDTSNLWGQGTLVTVSS

SEQ ID NO: 30 - protein, 119 aa, synthetic construct
  REGION 1..119: note = humanized antibody
EVQLEESGGDLVQPGGSLRLSCAASGFSFSFGYYMCWVRQAPGKGLEWVACIYGDSSDTLYANWAKGRFTISKTNSKNTLYLQMNSLRAEDTAVYFCARYPGGSYYNLWGQGTLVTVSS

SEQ ID NO: 31 - protein, 120 aa, synthetic construct
  REGION 1..120: note = humanized antibody
QVQLQESGGDLVQPGASLRLSCAASGFSFSSTYYMCWVRQAPGKGLEWVACIYAGSSGSTYYASWAKGRFTISKNSSTLYLQMNSLRAEDTAVYFCARVDGSSSGSWDLWGQGTLVTVSS

SEQ ID NO: 32 - protein, 121 aa, synthetic construct
  REGION 1..121: note = humanized antibody
EVQLEESGGDLVQPGASLRLSCAASGISFSSSDFMCWVRQAPGKGLEWVACIYAGSSVSIYYATWAKGRFTISKASSTTLYLQMNSLRAEDTAVYFCARSTGSVGRGFNLWGQGTLVTVSS

SEQ ID NO: 33 - protein, 121 aa, synthetic construct
  REGION 1..121: note = humanized antibody
EVQLVESGGGLVQPGGSLRLSCAASGFSFSSIYYMCWVRQAPGKGLEWVGCIYTGNSDFTYYANWAKGRFTISRDNSKSTLYLQMNSLRAEDTAVYFCARFRDDYASLKLWGQGTLVTVSS

SEQ ID NO: 34 - protein, 120 aa, synthetic construct
  REGION 1..120: note = humanized antibody
QVQLQESGGGLVQPGGSLRLSCTASGFSFSSGYDMCWVRQAPGKGLEWVGCIYTGSGSTYYANWAKGRFTISKDNSKTTLYLQMNSLRAEDTAVYFCARNSNDWMYFNLWGQGTLVTVSS

SEQ ID NO: 35 - protein, 110 aa, synthetic construct
  REGION 1..110: note = humanized antibody
DIVMTQSPSSLSASVGDRVTITCQASESISNYLSWYQQKPGQAPKLLIYLASTLASGVPSRFSGSGSGTDFTLTISSLQPEDFATYYCQNWWVIEHNGAAFGGGTKVVIK

SEQ ID NO: 36 - protein, 110 aa, synthetic construct
  REGION 1..110: note = humanized antibody
DIQMTQSPSSLSASVGDRVTITCQASESIYSNLAWYQQKPGQAPKLLIYAASLLASGVPSRFSGSGSGTDFTLTISSLQPEDFATYYCQSASYSTGPDWTFGQGTKVVIK

SEQ ID NO: 37 - protein, 110 aa, synthetic construct
  REGION 1..110: note = humanized antibody
DIQMTQSPSSLSASVGDRVTITCQASQSIYIYLSWYQQKPGQAPKLLIYDASKLASGVPSRFSGSGSGTDFTLTISSLQPEDFATYYCQQGATTYNVDNVFGQGTKVVIK

SEQ ID NO: 38 - protein, 111 aa, synthetic construct
  REGION 1..111: note = humanized antibody
DIVMTQSPSSLSASVGDRVTITCQASENIGNGLAWYQQKPGQAPKLLIYGASTLASGVPSRFSGSGSGTDFTLTISSLQPEDFATYYCQCTYWNPDYIGGAFGGGTKVVIK

SEQ ID NO: 39 - protein, 110 aa, synthetic construct
  REGION 1..110: note = humanized antibody
DIQMTQSPSSLSASVGDRVTITCLASEDIYSGISWYQQKPGKAPKLLIYAASNLESGVPSRFSGSGSGTDFTLTISSLQPEDFATYYCLGGYSYSNTGPTFGQGTKVEIK

SEQ ID NO: 40 - protein, 113 aa, synthetic construct
  REGION 1..113: note = humanized antibody
DIVMTQSPSSLSASVGDRVTITCQASEDIYSNLAWFQQKPGQAPKLLIYGASTLASGVPSRFSGSGSGTEFTLTISSLQPEDFATYYCLGVCTDISTDDLYNAFGQGTKVVIK

SEQ ID NO: 41 - protein, 110 aa, synthetic construct
  REGION 1..110: note = humanized antibody
DIVMTQSPSSLSASVGDRVTITCQASEDIYSNLAWFQQKPGQAPKLLIYRASTLASGVPSRFSGSGSGTEFTLTISSLQPEDFATYYCLGVYTYSTDIHAFGGGTKVVIK

SEQ ID NO: 42 - protein, 110 aa, synthetic construct
  REGION 1..110: note = humanized antibody
DIVMTQSPSSLSASVGDRVTITCQASEDIYSNLAWFQQKPGQAPKLLIYDASTLASGVPSRFSGSGSGTEFTLTISSLQPEDFATYYCLGVYTHISADNAFGGGTKVVIK

SEQ ID NO: 43 - protein, 111 aa, synthetic construct
  REGION 1..111: note = humanized antibody
DIQMTQSPSSLSASVGDRVTITCQASENIYSSLAWYQQKPGQAPKLLIYDASDLASGVPSRFSGSGSGTDFTLTISSLQPEDFATYYCQQGYYSGGTDNDVFGGGTKVVIK

SEQ ID NO: 44 - protein, 112 aa, synthetic construct
  REGION 1..112: note = humanized antibody
DIVMTQSPSSLSASVGDRVTITCQSSQSVDGNNLLSWYQQKPGQAPKLLIYDASNLASGVPSRFSGSGSGTDFTLTISSLQPEDFATYYCQGSYYSSSWYNVFGQGTKVVIK

SEQ ID NO: 45 - protein, 108 aa, synthetic construct
  REGION 1..108: note = humanized antibody
DIQMTQSPSSLSASVGDRVTITCQASQSIYSFLSWYQQKPGQAPKLLIYAASDLESGVPSRFSGSGSGTDFTLTISSLQPEDFATYYCQCNYIIDYGAFGQGTKVVIK

SEQ ID NO: 46 - protein, 108 aa, synthetic construct
  REGION 1..108: note = humanized antibody
DIQMTQSPSSLSASVGDRVTITCQASQSIGYYLAWYQQKPGQAPKLLIYRASTLASGVPSRFSGSGSGTDFTLTISSLQPEDFATYYCQSYYNSDSDAFGQGTKVVIK

SEQ ID NO: 47 - protein, 110 aa, synthetic construct
  REGION 1..110: note = humanized antibody
DIVMTQSPSSLSASVGDRVTITCQASQTISINLAWYQQKPGQAPKLLIYYASTLASGVPSRFSGSGSGTDFTLTISSLQPEDFATYYCQQGYTEDNIDNTFGQGTKVVIK

SEQ ID NO: 48 - protein, 110 aa, synthetic construct
  REGION 1..110: note = humanized antibody
DIQMTQSPSSLSASVGDRVTITCQASQNIYSNLAWYQQKPGQAPKLLIYAASLLASGVPSRFSGSGSGTDFTLTISSLQPEDFATYYCQGAVYSGNTEWAFGQGTKVVIK

SEQ ID NO: 49 - protein, 113 aa, synthetic construct
  REGION 1..113: note = humanized antibody
DIVMTQSPSSLSASVGDRVTITCQASQSVYNSNHLSWYQQKPGQAPKLLIYSASTLASGVPSRFSGSGSGTDFTLTISSLQPEDFATYYCQGEFSCVSADCIAFGGGTKVVIK

SEQ ID NO: 50 - protein, 110 aa, synthetic construct
  REGION 1..110: note = humanized antibody
DIQMTQSPSSLSASVGDRVTITCQASQSISSYLSWYQQKPGQAPKLLIYGASNLASGVPSRFSGSGSGTDFTLTISSLQPEDFATYYCQCTYYDNNYGGAFGGGTKVVIK

SEQ ID NO: 51 - protein, 114 aa, synthetic construct
  REGION 1..114: note = humanized antibody
DIVMTQSPSSLSASVGDRVTITCQASESISANYWSWYQQKPGQAPKLLIYGASTLASGVPSRFSGSGSGTDFTLTISSLQPEDFATYFCQSWYYSGSGSYHSWAFGQGTKVVIK

SEQ ID NO: 52 - protein, 110 aa, synthetic construct; identical to SEQ ID NO: 35
  REGION 1..110: note = humanized antibody

SEQ ID NO: 53 - protein, 110 aa, synthetic construct
  REGION 1..110: note = humanized antibody
AIQMTQSPSSLSASVGDRVTITCQASESIYSNLAWYQQKPGQAPKLLIYAASLLASGVPSRFSGSGSGTDYTLTISSLQPEDFATYYCQSASYSTGPDWTFGQGTKVVIK

SEQ ID NO: 54 - protein, 110 aa, synthetic construct
  REGION 1..110: note = humanized antibody
AIRMTQSPSSLSASVGDRVTITCQASQSIYIYLSWYQQKPGQAPKLLIYDASKLASGVPSRFSGSGSGTDFTLTISSLQPEDFATYYCQQGATTYNVDNVFGQGTKVVIK

SEQ ID NO: 55 - protein, 111 aa, synthetic construct
  REGION 1..111: note = humanized antibody
ELVMTQSPSSLSASVGDRVTITCQASENIGNGLAWYQQKPGQAPKLLIYGASTLASGVPSRFSGSGSGTDFTLTISSLQPEDFATYYCQCTYWNPDYIGGAFGGGTKVVIK

SEQ ID NO: 56 - protein, 110 aa, synthetic construct
  REGION 1..110: note = humanized antibody
AIQMTQSPSSLSASVGDRVTITCLASEDIYSGISWYQQKPGKAPKLLIYAASNLESGVPSRFSGSGSGTDYTLTISSLQPEDFATYYCLGGYSYSNTGPTFGQGTKVEIK

SEQ ID NO: 57 - protein, 113 aa, synthetic construct; identical to SEQ ID NO: 40
  REGION 1..113: note = humanized antibody

SEQ ID NO: 58 - protein, 110 aa, synthetic construct; identical to SEQ ID NO: 41
  REGION 1..110: note = humanized antibody

SEQ ID NO: 59 - protein, 110 aa, synthetic construct
  REGION 1..110: note = humanized antibody
ELVMTQSPSSLSASVGDRVTITCQASEDIYSNLAWFQQKPGQAPKLLIYDASTLASGVPSRFSGSGSGTEFTLTISSLQPEDFATYYCLGVYTHISADNAFGGGTKVEIK

SEQ ID NO: 60 - protein, 111 aa, synthetic construct
  REGION 1..111: note = humanized antibody
ALQMTQSPSSLSASVGDRVTITCQASENIYSSLAWYQQKPGQAPKLLIYDASDLASGVPSRFSGSGSGTDFTLTISSLQPEDFATYYCQQGYYSGGTDNDVFGGGTKVVIK

SEQ ID NO: 61 - protein, 112 aa, synthetic construct
  REGION 1..112: note = humanized antibody
NIVMTQSPSSLSASVGDRVTITCQSSQSVDGNNLLSWYQQKPGQAPKLLIYDASNLASGVPSRFSGSGSGTDFTLTISSLQPEDFATYYCQGSYYSSSWYNVFGQGTKVVIK

SEQ ID NO: 62 - protein, 108 aa, synthetic construct
  REGION 1..108: note = humanized antibody
DVQMTQSPSSLSASVGDRVTITCQASQSIYSFLSWYQQKPGQAPKLLIYAASDLESGVPSRFSGSGSGTDFTLTISSLQPEDFATYYCQCNYIIDYGAFGQGTKVVIK

SEQ ID NO: 63 - protein, 108 aa, synthetic construct; identical to SEQ ID NO: 46
  REGION 1..108: note = humanized antibody

SEQ ID NO: 64 - protein, 110 aa, synthetic construct
  REGION 1..110: note = humanized antibody
AIVMTQSPSSLSASVGDRVTITCQASQTISINLAWYQQKPGQAPKLLIYYASTLASGVPSRFSGSGSGTDFTLTISSLQPEDFATYYCQQGYTEDNIDNTFGQGTKVVIK

SEQ ID NO: 65 - protein, 110 aa, synthetic construct
  REGION 1..110: note = humanized antibody
AIQMTQSPSSLSASVGDRVTITCQASQNIYSNLAWYQQKPGQAPKLLIYAASLLASGVPSRFSGSGSGTDYTLTISSLQPEDFATYYCQGAVYSGNTEWAFGQGTKVVIK

SEQ ID NO: 66 - protein, 113 aa, synthetic construct; identical to SEQ ID NO: 49
  REGION 1..113: note = humanized antibody

SEQ ID NO: 67 - protein, 110 aa, synthetic construct
  REGION 1..110: note = humanized antibody
DVVMTQSPSSLSASVGDRVTITCQASQSISSYLSWYQQKPGQAPKLLIYGASNLASGVPSRFSGSGSGTDFTLTISSLQPEDFATYYCQCTYYDNNYGGAFGGGTKVEIK

SEQ ID NO: 68 - protein, 114 aa, synthetic construct; identical to SEQ ID NO: 51
  REGION 1..114: note = humanized antibody

SEQ ID NO: 69 - protein, 6 aa, synthetic construct
  REGION 1..6: note = humanized antibody
SSYWIC

SEQ ID NO: 70 - protein, 6 aa, synthetic construct
  REGION 1..6: note = humanized antibody
SSHYMC

SEQ ID NO: 71 - protein, 5 aa, synthetic construct
  REGION 1..5: note = humanized antibody
SYAMG

SEQ ID NO: 72 - protein, 5 aa, synthetic construct
  REGION 1..5: note = humanized antibody
NYAMG

SEQ ID NO: 73 - protein, 5 aa, synthetic construct
  REGION 1..5: note = humanized antibody
SYYMS

SEQ ID NO: 74 - protein, 6 aa, synthetic construct
  REGION 1..6: note = humanized antibody
SNYWIC

SEQ ID NO: 75 - protein, 6 aa, synthetic construct
  REGION 1..6: note = humanized antibody
SSYYMC

SEQ ID NO: 76 - protein, 6 aa, synthetic construct
  REGION 1..6: note = humanized antibody
TSYWRC

SEQ ID NO: 77 - protein, 6 aa, synthetic construct
  REGION 1..6: note = humanized antibody
SYYYMC

SEQ ID NO: 78 - protein, 5 aa, synthetic construct
  REGION 1..5: note = humanized antibody
SYWMS

SEQ ID NO: 79 - protein, 5 aa, synthetic construct
  REGION 1..5: note = humanized antibody
SYDMS

SEQ ID NO: 80 - protein, 6 aa, synthetic construct
  REGION 1..6: note = humanized antibody
SNYYMC

SEQ ID NO: 81 - protein, 6 aa, synthetic construct
  REGION 1..6: note = humanized antibody
FGYYMC

SEQ ID NO: 82 - protein, 6 aa, synthetic construct
  REGION 1..6: note = humanized antibody
STYYMC

SEQ ID NO: 83 - protein, 6 aa, synthetic construct
  REGION 1..6: note = humanized antibody
SSDFMC

SEQ ID NO: 84 - protein, 6 aa, synthetic construct
  REGION 1..6: note = humanized antibody
SIYYMC

SEQ ID NO: 85 - protein, 6 aa, synthetic construct
  REGION 1..6: note = humanized antibody
SGYDMC

SEQ ID NO: 86 - protein, 17 aa, synthetic construct
  REGION 1..17: note = humanized antibody
CIYTGSGGTYYASWEKG

SEQ ID NO: 87 - protein, 18 aa, synthetic construct
  REGION 1..18: note = humanized antibody
CIYAGSSGNTYYANWAKG

SEQ ID NO: 88 - protein, 16 aa, synthetic construct
  REGION 1..16: note = humanized antibody
VITSSATTYYASWAKG

SEQ ID NO: 89 - protein, 16 aa, synthetic construct
  REGION 1..16: note = humanized antibody
VISSDGFFYDASWAKG

SEQ ID NO: 90 - protein, 16 aa, synthetic construct
  REGION 1..16: note = humanized antibody
IISGSASTYYATWAKG

SEQ ID NO: 91 - protein, 17 aa, synthetic construct
  REGION 1..17: note = humanized antibody
CIYTSTGNTWYASWAKG

SEQ ID NO: 92 - protein, 18 aa, synthetic construct
  REGION 1..18: note = humanized antibody
CIYAGSSGVTYYASWAKG

SEQ ID NO: 93 - protein, 18 aa, synthetic construct
  REGION 1..18: note = humanized antibody
CIYAGSGDVTYYANWVNG

SEQ ID NO: 94 - protein, 18 aa, synthetic construct
  REGION 1..18: note = humanized antibody
CIFIGYGDVTWYASWAKG

SEQ ID NO: 95 - protein, 16 aa, synthetic construct
  REGION 1..16: note = humanized antibody
MIYGSGYTYYASWAKG

SEQ ID NO: 96 - protein, 16 aa, synthetic construct
  REGION 1..16: note = humanized antibody
TIYIGGTTAYASWPKG

SEQ ID NO: 97 - protein, 17 aa, synthetic construct
  REGION 1..17: note = humanized antibody
CIYTNSGNTWSASWAKG

SEQ ID NO: 98 - protein, 17 aa, synthetic construct
  REGION 1..17: note = humanized antibody
CIYGDSSDTLYANWAKG

SEQ ID NO: 99 - protein, 18 aa, synthetic construct
  REGION 1..18: note = humanized antibody
CIYAGSSGSTYYASWAKG

SEQ ID NO: 100 - protein, 18 aa, synthetic construct
  REGION 1..18: note = humanized antibody
CIYAGSSVSIYYATWAKG

SEQ ID NO: 101 - protein, 18 aa, synthetic construct
  REGION 1..18: note = humanized antibody
CIYTGNSDFTYYANWAKG

SEQ ID NO: 102 - protein, 17 aa, synthetic construct
  REGION 1..17: note = humanized antibody
CIYTGSGSTYYANWAKG

SEQ ID NO: 103 - protein, 8 aa, synthetic construct
  REGION 1..8: note = humanized antibody
DPGYSSWL

SEQ ID NO: 104 - protein, 11 aa, synthetic construct
  REGION 1..11: note = humanized antibody
VDASSSGSWDL

SEQ ID NO: 105 - protein, 15 aa, synthetic construct
  REGION 1..15: note = humanized antibody
GGPGYSTNTHYAFDP

SEQ ID NO: 106 - protein, 11 aa, synthetic construct
  REGION 1..11: note = humanized antibody
DRGTSTGSLDL

SEQ ID NO: 107 - protein, 17 aa, synthetic construct
  REGION 1..17: note = humanized antibody
THYAAVAGYGYASRLDL

SEQ ID NO: 108 - protein, 10 aa, synthetic construct
  REGION 1..10: note = humanized antibody
DLLVVTSFNL

SEQ ID NO: 109 - protein, 9 aa, synthetic construct
  REGION 1..9: note = humanized antibody
ETDGNYFNL

SEQ ID NO: 110 - protein, 9 aa, synthetic construct
  REGION 1..9: note = humanized antibody
GVGFGYFNL

SEQ ID NO: 111 - protein, 11 aa, synthetic construct
  REGION 1..11: note = humanized antibody
ALGSSGYRVNL

SEQ ID NO: 112 - protein, 7 aa, synthetic construct
  REGION 1..7: note = humanized antibody
DPQYFIL

SEQ ID NO: 113 - protein, 12 aa, synthetic construct
  REGION 1..12: note = humanized antibody
LQGANYYNSLAL

SEQ ID NO: 114 - protein, 10 aa, synthetic construct
  REGION 1..10: note = humanized antibody
DLNYPDTSNL

SEQ ID NO: 115 - protein, 9 aa, synthetic construct
  REGION 1..9: note = humanized antibody
YPGGSYYNL

SEQ ID NO: 116 - protein, 11 aa, synthetic construct
  REGION 1..11: note = humanized antibody
VDGSSSGSWDL

SEQ ID NO: 117 - protein, 11 aa, synthetic construct
  REGION 1..11: note = humanized antibody
STGSVGRGFNL

SEQ ID NO: 118 - protein, 10 aa, synthetic construct
  REGION 1..10: note = humanized antibody
FRDDYASLKL

SEQ ID NO: 119 - protein, 10 aa, synthetic construct
  REGION 1..10: note = humanized antibody
NSNDWMYFNL

SEQ ID NO: 120 - protein, 11 aa, synthetic construct
  REGION 1..11: note = humanized antibody
QASESISNYLS

SEQ ID NO: 121 - protein, 11 aa, synthetic construct
  REGION 1..11: note = humanized antibody
QASESIYSNLA

SEQ ID NO: 122 - protein, 11 aa, synthetic construct
  REGION 1..11: note = humanized antibody
QASQSIYIYLS

SEQ ID NO: 123 - protein, 11 aa, synthetic construct
  REGION 1..11: note = humanized antibody
QASENIGNGLA

SEQ ID NO: 124 - protein, 11 aa, synthetic construct
  REGION 1..11: note = humanized antibody
LASEDIYSGIS

SEQ ID NO: 125 - protein, 11 aa, synthetic construct
  REGION 1..11: note = humanized antibody
QASEDIYSNLA

SEQ ID NO: 126 - protein, 11 aa, synthetic construct
  REGION 1..11: note = humanized antibody
QASEDIYSNLA

SEQ ID NO: 127 - protein, 11 aa, synthetic construct
  REGION 1..11: note = humanized antibody
QASEDIYSNLA

SEQ ID NO: 128 - protein, 11 aa, synthetic construct
  REGION 1..11: note = humanized antibody
QASENIYSSLA

SEQ ID NO: 129 - protein, 13 aa, synthetic construct
  REGION 1..13: note = humanized antibody
QSSQSVDGNNLLS

SEQ ID NO: 130 - protein, 11 aa, synthetic construct
  REGION 1..11: note = humanized antibody
QASQSIYSFLS

SEQ ID NO: 131 - protein, 11 aa, synthetic construct
  REGION 1..11: note = humanized antibody
QASQSIGYYLA

SEQ ID NO: 132 - protein, 11 aa, synthetic construct
  REGION 1..11: note = humanized antibody
QASQTISINLA

SEQ ID NO: 133 - protein, 11 aa, synthetic construct
  REGION 1..11: note = humanized antibody
QASQNIYSNLA

SEQ ID NO: 134 - protein, 13 aa, synthetic construct
  REGION 1..13: note = humanized antibody
QASQSVYNSNHLS

SEQ ID NO: 135 - protein, 11 aa, synthetic construct
  REGION 1..11: note = humanized antibody
QASQSISSYLS

SEQ ID NO: 136 - protein, 12 aa, synthetic construct
  REGION 1..12: note = humanized antibody
QASESISANYWS

SEQ ID NO: 137 - protein, 7 aa, synthetic construct
  REGION 1..7: note = humanized antibody
LASTLAS

SEQ ID NO: 138 - protein, 7 aa, synthetic construct
  REGION 1..7: note = humanized antibody
AASLLAS

SEQ ID NO: 139 - protein, 7 aa, synthetic construct
  REGION 1..7: note = humanized antibody
DASKLAS

SEQ ID NO: 140 - protein, 7 aa, synthetic construct
  REGION 1..7: note = humanized antibody
GASTLAS

SEQ ID NO: 141 - protein, 7 aa, synthetic construct
  REGION 1..7: note = humanized antibody
AASNLES

SEQ ID NO: 142 - protein, 7 aa, synthetic construct
  REGION 1..7: note = humanized antibody
GASTLAS

SEQ ID NO: 143 - protein, 7 aa, synthetic construct
  REGION 1..7: note = humanized antibody
RASTLAS

SEQ ID NO: 144 - protein, 7 aa, synthetic construct
  REGION 1..7: note = humanized antibody
DASTLAS

SEQ ID NO: 145 - protein, 7 aa, synthetic construct
  REGION 1..7: note = humanized antibody
DASDLAS

SEQ ID NO: 146 - protein, 7 aa, synthetic construct
  REGION 1..7: note = humanized antibody
DASNLAS

SEQ ID NO: 147 - protein, 7 aa, synthetic construct
  REGION 1..7: note = humanized antibody
AASDLES

SEQ ID NO: 148 - protein, 7 aa, synthetic construct
  REGION 1..7: note = humanized antibody
RASTLAS

SEQ ID NO: 149 - protein, 7 aa, synthetic construct
  REGION 1..7: note = humanized antibody
YASTLAS

SEQ ID NO: 150 - protein, 7 aa, synthetic construct
  REGION 1..7: note = humanized antibody
AASLLAS

SEQ ID NO: 151 - protein, 7 aa, synthetic construct
  REGION 1..7: note = humanized antibody
SASTLAS

SEQ ID NO: 152 - protein, 7 aa, synthetic construct
  REGION 1..7: note = humanized antibody
GASNLAS

SEQ ID NO: 153 - protein, 7 aa, synthetic construct
  REGION 1..7: note = humanized antibody
GASTLAS

SEQ ID NO: 154 - protein, 12 aa, synthetic construct
  REGION 1..12: note = humanized antibody
QNWWVIEHNGAA

SEQ ID NO: 155 - protein, 12 aa, synthetic construct
  REGION 1..12: note = humanized antibody
QSASYSTGPDWT

SEQ ID NO: 156 - protein, 12 aa, synthetic construct
  REGION 1..12: note = humanized antibody
QQGATTYNVDNV

SEQ ID NO: 157 - protein, 13 aa, synthetic construct
  REGION 1..13: note = humanized antibody
QCTYWNPDYIGGA

SEQ ID NO: 158 - protein, 12 aa, synthetic construct
  REGION 1..12: note = humanized antibody
LGGYSYSNTGPT

SEQ ID NO: 159 - protein, 15 aa, synthetic construct
  REGION 1..15: note = humanized antibody
LGVCTDISTDDLYNA

SEQ ID NO: 160 - protein, 12 aa, synthetic construct
  REGION 1..12: note = humanized antibody
LGVYTYSTDIHA

SEQ ID NO: 161 - protein, 12 aa, synthetic construct
  REGION 1..12: note = humanized antibody
LGVYTHISADNA

SEQ ID NO: 162 - protein, 13 aa, synthetic construct
  REGION 1..13: note = humanized antibody
QQGYYSGGTDNDV

SEQ ID NO: 163 - protein, 12 aa, synthetic construct
  REGION 1..12: note = humanized antibody
QGSYYSSSWYNV

SEQ ID NO: 164 - protein, 10 aa, synthetic construct
  REGION 1..10: note = humanized antibody
QCNYIIDYGA

SEQ ID NO: 165 - protein, 10 aa, synthetic construct
  REGION 1..10: note = humanized antibody
QSYYNSDSDA

SEQ ID NO: 166 - protein, 12 aa, synthetic construct
  REGION 1..12: note = humanized antibody
QQGYTEDNIDNT

SEQ ID NO: 167 - protein, 12 aa, synthetic construct
  REGION 1..12: note = humanized antibody
QGAVYSGNTEWA

SEQ ID NO: 168 - protein, 13 aa, synthetic construct
  REGION 1..13: note = humanized antibody
QGEFSCVSADCIA

SEQ ID NO: 169 - protein, 12 aa, synthetic construct
  REGION 1..12: note = humanized antibody
QCTYYDNNYGGA

SEQ ID NO: 170 - protein, 15 aa, synthetic construct
  REGION 1..15: note = humanized antibody
QSWYYSGSGSYHSWA

SEQ ID NO: 171 - protein, 107 aa, Homo sapiens [sequence omitted]

SEQ ID NO: 172 - protein, 329 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 173 - protein, 123 aa, synthetic construct
  REGION 1..123: note = humanized antibody
EVQLLESGGRLVQPGTSLRLSCAVSGIDLSSYAMGWVRQAPGKGLEYVGVITSSATTYYASWAKGRFTISKTSSKNTLYLQMNSLRAEDTAVYFCARGGPGYSTNTHYAFDPWGQGTLVTVSS

SEQ ID NO: 174 - protein, 108 aa, synthetic construct
  REGION 1..108: note = Humanized antibody
DVQMTQSPSSLSASVGDRVTITCQASQSIYSFLSWYQQKPGQAPKLLIYAASDLESGVPSRFSGSGSGTDFTLTISSLQPEDFATYYCQSNYIIDYGAFGQGTKVVIK

SEQ ID NO: 175 - protein, 10 aa, synthetic construct
  REGION 1..10: note = Humanized antibody
QSNYIIDYGA